(19) United States
(12) Patent Application Publication
(10) Pub. No.: US 2025/0283150 A1
(43) Pub. Date: Sep. 11, 2025

(54) NUCLEIC ACID STRUCTURE

(71) Applicants: CRANEBIO CO., LTD., Tokyo (JP); THE SCHOOL CORPORATION KANSAI UNIVERSITY

(72) Inventors: Akinori KUZUYA, Suita-shi, Osaka (JP); Nozomu TAKAHASHI, Suita-shi, Osaka (JP); Yuta OKAMOTO, Suita-shi, Osaka (JP); Sachiko KONO, Ibaraki-shi, Osaka (JP); Chihiro KATO, Osaka-shi, Osaka (JP); Keita SAITO, Tokyo (JP)

(21) Appl. No.: 18/019,375

(22) PCT Filed: Jul. 30, 2021
(86) PCT No.: PCT/JP2021/028281

§ 371 Date: Feb. 2, 2023

(30) Foreign Application Priority Data
Aug. 5, 2020 (JP) 2020-132829

(51) Int. Cl. C12Q 1/6816; C12N 15/115

(52) U.S. Cl. CPC C12Q 1/6816; C12N 15/115; C12N 2310/16

(57) ABSTRACT

An object is to provide a technique for detecting a test substance more easily using a nucleic acid structure. The object is achieved by a nucleic acid structure comprising a partial nucleic acid structure 1 and a partial nucleic acid structure 2, the partial nucleic acid structure 1 and the partial nucleic acid structure 2 being linked by a Holliday junction, the partial nucleic acid structure 1 comprising a protruding structure 1a containing a nucleic acid aptamer sequence, and a protruding structure 1b containing a split domain 1 of an enzyme, and the partial nucleic acid structure 2 comprising a protruding structure 2a containing a sequence complementary to part or all of the nucleic acid aptamer sequence, and a protruding structure 2b containing a split domain 2 paired with the split domain 1.

### CROSS-REFERENCE TO RELATED APPLICATIONS

This application is a U.S. National Stage application of PCT/JP2021/028281 filed 30 Jul. 2021, which claims priority to Japanese application No. 2020-132829 filed 5 Aug. 2020, the entire disclosures of which are hereby incorporated by reference in their entireties.

### SEQUENCE LISTING

The present application contains a Sequence Listing which has been submitted electronically in ASCII format and is hereby incorporated by reference in its entirety. Said ASCII copy, created on 11 Jun. 2023, is named 2023-06-11SEQUENCELISTING.txt and is 83 kilobytes in size.

### TECHNICAL FIELD

The present invention relates to a nucleic acid structure and the like.

### BACKGROUND ART

Techniques for detecting nucleic acids, proteins, peptides, low-molecular-weight compounds, metal ions, and the like are used in various fields, such as medical and environmental fields. For example, disease tests may be performed using the presence and amount of specific nucleic acids and other biomolecules as indicators.

On the other hand, DNA origami is a nucleic acid structure formed by folding single-stranded circular DNA into many short DNA strands (staple DNAs) that can complementarily bind to the DNA. Various structures can be created at will, depending on the design of the sequence of staple DNA. NPL 1 has reported DNA origami (DNA origami pliers (or DNA pliers)) having a structure in which two levers are joined at a fulcrum (Holliday junction). DNA origami pliers include three possible structures (FIG. 9: antiparallel, cross, and parallel types) based on the conformation of the Holliday junction. So far, there have been reports on techniques for detecting Na+, Ag+, streptavidin, IgG, ATP, and miRNA using structural changes of DNA origami pliers. However, it is necessary for these techniques to fluorescently detect small fluorescence intensity changes based on structural changes, or to detect structural changes by an atomic force microscope.

### CITATION LIST

### Non-Patent Literature

NPL 1: Kuzuya, A., (2011) Nat. Commun. doi: 10.1038/ncomms1452

### SUMMARY OF INVENTION

### Technical Problem

An object of the present invention is to provide a technique for detecting a test substance more easily using a nucleic acid structure.

### Solution to Problem

As a result of intensive research in view of the above object, the present inventors found that the object can be achieved by a nucleic acid structure comprising a partial nucleic acid structure 1 and a partial nucleic acid structure 2, the partial nucleic acid structure 1 and the partial nucleic acid structure 2 being linked by a Holliday junction, the partial nucleic acid structure 1 comprising a protruding structure 1a containing a nucleic acid aptamer sequence, and a protruding structure 1b containing a split domain 1 of an enzyme, and
the partial nucleic acid structure 2 comprising a protruding structure 2a containing a sequence complementary to part or all of the nucleic acid aptamer sequence, and a protruding structure 2b containing a split domain 2 paired with the split domain 1.
   Upon further research based on this finding, the present inventors have completed the present invention. More specifically, the present invention includes the following embodiments.

Item 1. A nucleic acid structure comprising a partial nucleic acid structure 1 and a partial nucleic acid structure 2,
the partial nucleic acid structure 1 and the partial nucleic acid structure 2 being linked by a Holliday junction,
the partial nucleic acid structure 1 comprising a protruding structure 1a containing a nucleic acid aptamer sequence, and a protruding structure 1b containing a split domain 1 of an enzyme, and
the partial nucleic acid structure 2 comprising a protruding structure 2a containing a sequence complementary to part or all of the nucleic acid aptamer sequence, and a protruding structure 2b containing a split domain 2 paired with the split domain 1.

Item 2. The nucleic acid structure according to Item 1, wherein the partial structure 1 and the partial structure 2 each have an elongated shape.

Item 3. The nucleic acid structure according to Item 1 or 2, which is made of a material containing a single-stranded circular nucleic acid and a staple nucleic acid containing a sequence complementary to the single-stranded circular nucleic acid.

Item 4. The nucleic acid structure according to any one of Items 1 to 3, wherein the protruding structures are arranged so that, among three structures (structural type 1, structural type 2, and structural type 3: provided that the structural type 2 is an intermediate structure that mediates structural changes between the structural type 1 and the structural type 3) based on the conformation of the Holliday junction, when taking the structural type 1, the protruding structure 1a and the protruding structure 2a face each other, and when taking the structural type 3, the protruding structure 1b and the protruding structure 2b face each other.

Item 5. The nucleic acid structure according to Item 4, wherein the protruding structures are arranged so that the protruding structure 1a and the protruding structure 2a face each other and bind together based on complementary base pairing in the absence of a recognition substance for the nucleic acid aptamer sequence.

Item 6. The nucleic acid structure according to Item 4 or 5, wherein the protruding structures are arranged so that the protruding structure 1b and the protruding structure 2b face each other to form the enzyme in the presence of a recognition substance for the nucleic acid aptamer sequence and in the presence of a monovalent cation.

Item 7. The nucleic acid structure according to any one of Items 1 to 6, wherein the enzyme is a nucleic acid enzyme containing two or more G-quartets.

Item 8. The nucleic acid structure according to any one of Items 1 to 7, wherein the recognition substance for the nucleic acid aptamer sequence is a biological material.

Item 9. The nucleic acid structure according to any one of Items 1 to 8, wherein the recognition substance for the nucleic acid aptamer sequence is at least one member selected from the group consisting of nucleic acids, proteins, peptides, low-molecular-weight compounds, physiologically active substances, and metal ions.

Item 10. The nucleic acid structure according to any one of Items 1 to 9, which is a nano-structure.

Item 11. A test substance detection composition comprising the nucleic acid structure according to any one of Items 1 to 10.

Item 12. The test substance detection composition according to Item 11, which is a reagent or a test agent.

Item 13. A method for detecting a test substance, comprising bringing the nucleic acid structure according to any one of Items 1 to 10 into contact with a sample that may contain the test substance.

### Advantageous Effects of Invention

According to the present invention, a technique for detecting a test substance more easily using a nucleic acid structure can be provided.

### BRIEF DESCRIPTION OF DRAWINGS

FIG. 1 shows the overall view of the nucleic acid structure of Reference Example 1.

FIG. 2 shows the leftmost diagram among the six diagrams obtained by dividing FIG. 1 into six parts. The sequences of single-stranded circular DNA and staple DNAs, and staple numbers are shown. The marker parts indicate staple DNAs. The black bar indicates the boundary with the adjacent divided diagram.

FIG. 3 shows the second diagram from the left among the six diagrams obtained by dividing FIG. 1 into six parts. The sequences of single-stranded circular DNA and staple DNAs, and staple numbers are shown. The marker parts indicate staple DNAs. The black bar indicates the boundary with the adjacent divided diagram.

FIG. 4 shows the third diagram from the left among the six diagrams obtained by dividing FIG. 1 into six parts. The sequences of single-stranded circular DNA and staple DNAs, and staple numbers are shown. The marker parts indicate staple DNAs. The black bar indicates the boundary with the adjacent divided diagram.

FIG. 5 shows the fourth diagram from the left among the six diagrams obtained by dividing FIG. 1 into six parts. The sequences of single-stranded circular DNA and staple DNAs, and staple numbers are shown. The marker parts indicate staple DNAs. The black bar indicates the boundary with the adjacent divided diagram.

FIG. 6 shows the fifth diagram from the left among the six diagrams obtained by dividing FIG. 1 into six parts. The sequences of single-stranded circular DNA and staple DNAs, and staple numbers are shown. The marker parts indicate staple DNAs. The black bar indicates the boundary with the adjacent divided diagram.

FIG. 7 shows the sixth (rightmost) diagram from the left among the six diagrams obtained by dividing FIG. 1 into six parts. The sequences of single-stranded circular DNA and staple DNAs, and staple numbers are shown. The marker parts indicate staple DNAs. The black bar indicates the boundary with the adjacent divided diagram.

FIG. 8 shows a schematic diagram showing only single-stranded circular DNA in a linear form in the nucleic acid structure of Reference Example 1.

FIG. 9 shows three structures based on the conformation of the Holliday junction in the nucleic acid structure of Reference Example 2.

FIG. 10 shows AFM observation images showing structural changes due to the addition of K⁺ in the nucleic acid structure of Reference Example 2.

FIG. 11 shows changes in the proportion of each structure due to the addition of K⁺ in the nucleic acid structure of Reference Example 2.

FIG. 12 shows a photographic image of the detection results in Reference Example 3.

FIG. 13 shows a schematic diagram of structural changes in the nucleic acid structure of Example 1.

FIG. 14 shows AFM observation images showing structural changes due to the addition of miR-20 in the nucleic acid structure of Example 1.

FIG. 15 shows changes in the proportion of each structure due to the addition of miR-20 in the nucleic acid structure of Example 1.

FIG. 16 shows a photographic image of the detection results in Example 2.

FIG. 17 shows a schematic diagram of structural changes in the nucleic acid structure of Example 3.

FIG. 18 shows AFM observation images showing structural changes due to the addition of R509 in the nucleic acid structure of Example 3.

FIG. 19 shows changes in the proportion of each structure due to the addition of R509 in the nucleic acid structure of Example 3.

FIG. 20 shows a photographic image of the detection results in Example 4.

FIG. 21 shows a schematic diagram of structural changes in the nucleic acid structure of Example 5.

FIG. 22 shows AFM observation images showing structural changes due to the addition of N1 in the nucleic acid structure of Example 5.

FIG. 23 shows changes in the proportion of each structure due to the addition of N1 in the nucleic acid structure of Example 5.

FIG. 24 shows a photographic image of the detection results in Example 6.

FIG. 25 shows a schematic diagram of structural changes in the nucleic acid structure of Example 7.

FIG. 26 shows AFM observation images showing structural changes due to the addition of thrombin in the nucleic acid structure of Example 7.

FIG. 27 shows changes in the proportion of each structure due to the addition of thrombin in the nucleic acid structure of Example 7.

FIG. 28 shows a photographic image of the detection results in Example 8.

FIG. 29 shows an electrophoretic image of LgBiT and LgBiT-138w of Example 9.

FIG. 30 shows a schematic diagram of structural changes in the nucleic acid structure of Example 9.

FIG. 31 shows AFM observation images showing structural changes due to the addition of miR-20 in the nucleic acid structure of Example 9.

FIG. 32 shows changes in the proportion of each structure due to the addition of miR-20 in the nucleic acid structure of Example 9.

FIG. 33 shows the detection results in Example 10.

### DESCRIPTION OF EMBODIMENTS

### 1. Definition

In the present specification, the terms “comprise” and “contain” include the concepts of “comprising,” “containing,” “consisting essentially of,” and “consisting of.”

In the present specification, the “identity” of base sequences refers to the degree of matching between two or more comparable base sequences. Therefore, the higher the match between two base sequences, the higher the identity or similarity of those sequences. The level of identity between base sequences is determined, for example, using a sequence analysis tool, FASTA, with default parameters. Alternatively, the identity level can be determined using the algorithm BLAST by Karlin and Altschul (Karlin S, Altschul SF. “Methods for assessing the statistical significance of molecular sequence features by using general scoring schemes” Proc Natl Acad Sci USA. 87:2264-2268 (1990), Karlin S, Altschul SF. “Applications and statistics for multiple high-scoring segments in molecular sequences.” Proc Natl Acad Sci USA. 90:5873-7 (1993)). A program called BLASTX has been developed based on the BLAST algorithm. Specific methods of these analysis methods are known, and reference may be made to the website of the National Center of Biotechnology Information (NCBI) (http://www.ncbi.nlm.nih.gov/).

In the present specification, nucleic acids, such as DNA and RNA, may be subjected to known chemical modification, as shown below. In order to prevent degradation by hydrolases, such as nucleases, the phosphate residue (phosphate) of each nucleotide can be replaced by a chemically modified phosphate residue, such as phosphorothioate (PS), methylphosphonate, or phosphorodithionate. The hydroxyl group at position 2 of the sugar (ribose) of each ribonucleotide may be replaced by —OR (R is, for example, CH3(2′-O-Me), CH2CH2OCH3(2′-O-MOE), CH2CH2NHC(NH)NH2, CH2CONHCH3, or CH2CH2CN). Further, a base moiety (pyrimidine, purine) may be chemically modified, and examples include introduction of a methyl group or a cationic functional group into position 5 of the pyrimidine base, substitution of the carbonyl group at position 2 with thiocarbonyl, and the like. Other examples include, but are not limited to, those in which the phosphate moiety or hydroxyl moiety is modified with biotin, an amino group, a lower alkylamine group, an acetyl group, or the like. In addition, BNA (LNA), in which the conformation of the sugar moiety is fixed at the N-type by bridging the 2′ oxygen and 4′ carbon of the sugar moiety of the nucleotide, can also be used.

### 2. Nucleic Acid Structure

The present invention relates to a nucleic acid structure comprising a partial nucleic acid structure 1 and a partial nucleic acid structure 2,
the partial nucleic acid structure 1 and the partial nucleic acid structure 2 being linked by a Holliday junction,
the partial nucleic acid structure 1 comprising a protruding structure 1a containing a nucleic acid aptamer sequence, and a protruding structure 1b containing a split domain 1 of an enzyme, and
the partial nucleic acid structure 2 comprising a protruding structure 2a containing a sequence complementary to part or all of the nucleic acid aptamer sequence, and a protruding structure 2b containing a split domain 2 paired with the split domain 1 (also referred to as “the nucleic acid structure of the present invention” in the present specification). This is described below.

The nucleic acid structure is not particularly limited as long as it mainly contains a nucleic acid. The content of the nucleic acid in the nucleic acid structure is, for example, 70 mass % or more, preferably 80 mass % or more, more preferably 90 mass % or more, even more preferably 95 mass % or more, still more preferably 98 mass % or more, still even more preferably 99 mass % or more, and particularly preferably 100 mass %. Substances other than the nucleic acid in the nucleic acid structure are not particularly limited, and examples include proteins, peptides, sugars, labeling substances, metal ions, and the like.

The nucleic acid structure may be a three-dimensional structure, a two-dimensional structure (planer structure), or a combination thereof.

The nucleic acid structure is preferably formed by folding a nucleic acid. In terms of being suitable for the formation of the nucleic acid structure of the present invention, the nucleic acid structure is preferably a structure (DNA origami) made of a material containing a single-stranded circular nucleic acid and a staple nucleic acid containing a sequence complementary to the single-stranded circular nucleic acid.

The single-stranded circular nucleic acid that can form the nucleic acid structure is not particularly limited as long as it can be folded to form DNA origami. A single-stranded circular nucleic acid that may be composed of any base sequence can form DNA origami by designing the sequence of staple DNA according thereto. The number of bases in the single-stranded circular nucleic acid is not particularly limited, and is, for example, 500 to 50000, preferably 1000 to 25000, more preferably 2000 to 15000, even more preferably 3000 to 12000, and still more preferably 4000 to 10000. The base sequence of the single-stranded circular nucleic acid is not particularly limited, and is, for example, a base sequence containing a M13 bacteriophage DNA sequence or a sequence derived from the DNA sequence. Specific examples of the base sequence include a base sequence containing a base sequence represented by SEQ ID NO: 273, and a base sequence containing a base sequence having 70% or more (preferably 80% or more, more preferably 90% or more, even more preferably 95% or more, still more preferably 98% or more, and still even more preferably 99% or more) identity to the base sequence represented by SEQ ID NO: 273.

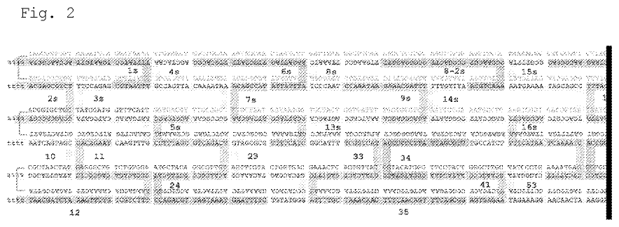

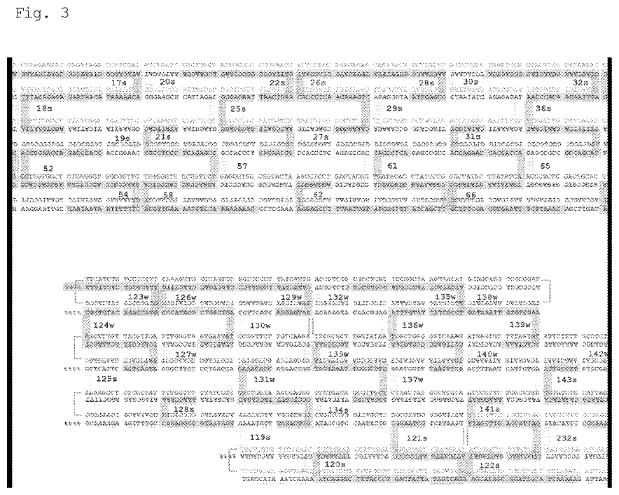

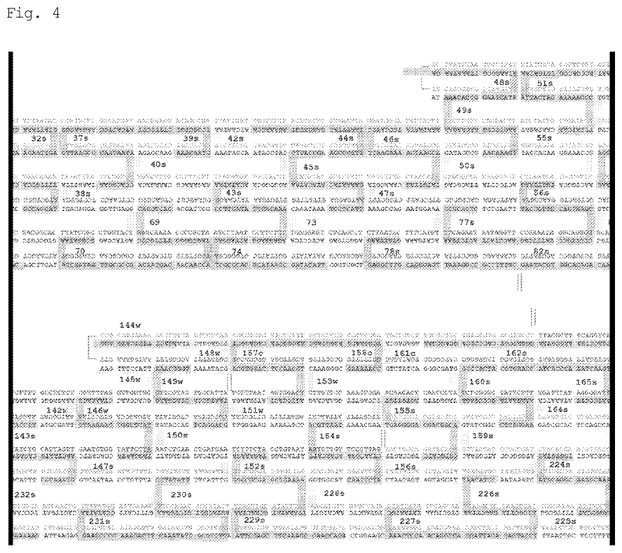

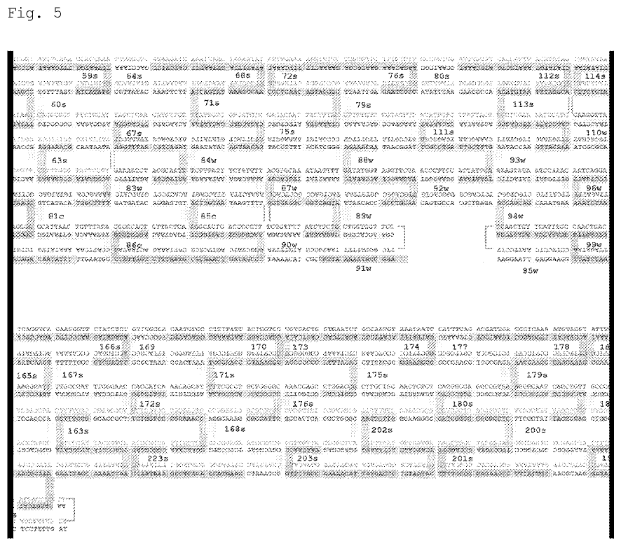

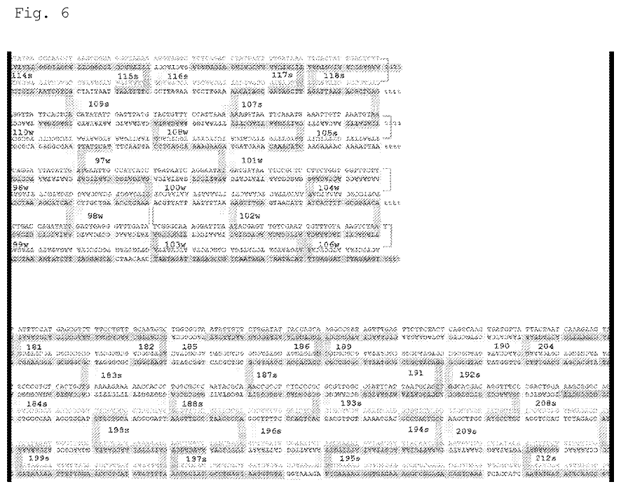

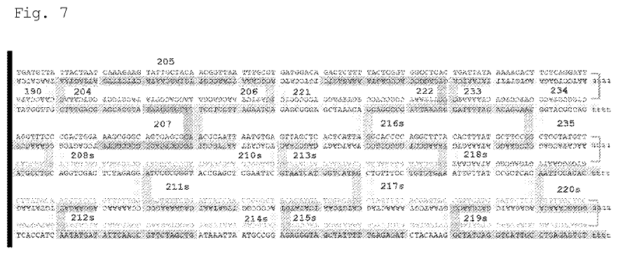

The staple nucleic acid that can form the nucleic acid structure is not particularly limited as long as it contains a sequence complementary to the single-stranded circular nucleic acid, and can fold the single-stranded circular nucleic acid by complementary base pairing with the single-stranded circular nucleic acid to form DNA origami. The number of bases in the staple nucleic acid is not particularly limited, and is, for example, 10 to 500, preferably 20 to 250, and more preferably 30 to 100. In the staple nucleic acid, the number of bases in the sequence complementary to the single-stranded circular nucleic acid is not particularly limited, and is, for example, 10 to 500, preferably 20 to 250, and more preferably 30 to 100. Like the staple DNAs indicated by markers in FIGS. 2 to 7, one staple nucleic acid generally contains sequences complementary to a plurality of (e.g., 2 to 5, preferably 2 to 4, and more preferably 2 or 3) adjacent strands that are present when a single-stranded circular nucleic acid is folded. A staple nucleic acid having such sequence characteristics can further stabilize the single-stranded circular nucleic acid in a folded state through complementary base pairing with the single-stranded circular nucleic acid. However, in the nucleic acid structure of the present invention, two staple nucleic acids involved in Holliday junction formation (82s and 162s in Reference Example 1, provided later) exceptionally, rather than forming a complementary base pair across a plurality of adjacent strands that are present when the single-stranded circular nucleic acid is folded, preferably form a complementary base pair with only one of these strands. Various DNA origami structures have been reported so far, and the sequence of staple nucleic acid for forming a nucleic acid structure of the desired shape and configuration can be designed according to known methods and information.

The size of the nucleic acid structure is not particularly limited, and can be suitably adjusted depending on the shape, size, etc. of the test substance. The nucleic acid structure is preferably nano-sized (nano-structure). For example, the longest diameter (major diameter) of the nucleic acid structure is 1000 nm or less. In an embodiment of the present invention, the major diameter of the nucleic acid structure is, for example, 5 to 1000 nm, preferably 20 to 800 nm, more preferably 50 to 600 nm, and even more preferably 100 to 400 nm.

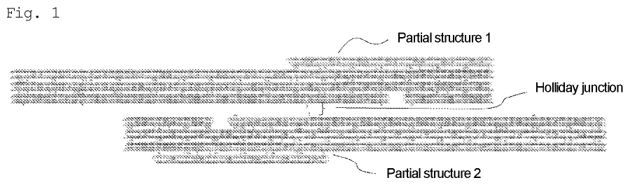

The nucleic acid structure contains a partial nucleic acid structure 1 and a partial nucleic acid structure 2. The partial nucleic acid structure 1 and the partial nucleic acid structure 2 are each part of the above nucleic acid structure, and linked to each other by a Holliday junction (e.g., FIG. 1). That is, the partial nucleic acid structure 1 has two of four arms that form the Holliday junction, and the partial nucleic acid structure 2 has the other two arms. In Reference Example 1, provided later, in the partial nucleic acid structure 1, double-stranded DNA formed by staple DNA with staple number 82s and a single-stranded circular nucleic acid forms two arms, and in the partial nucleic acid structure 2, double-stranded DNA formed by staple DNA with staple number 162s and a single-stranded circular nucleic acid forms the other two arms.

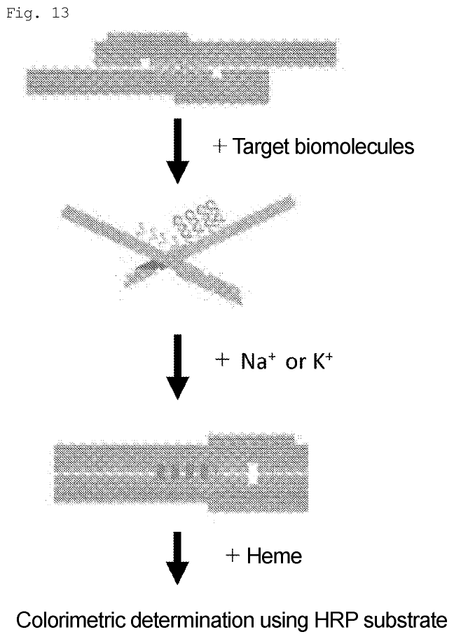

The nucleic acid structure can take three structures (structural type 1, structural type 2, and structural type 3) based on the conformation of the Holliday junction. The structural type 2 is an intermediate structure that mediates structural changes between the structural type 1 and the structural type 3. That is, the structural type 1 can be structurally changed to the structural type 3 via the structural type 2, and the structural type 3 can be structurally changed to the structural type 1 via the structural type 2 (e.g., FIG. 13). As in the Reference Examples and Examples provided later, when the structural type 1 and the structural type 3 differ in the shape of the nucleic acid structure, as an embodiment, the structural type 1 can be expressed as antiparallel type (or parallel type), and the structural type 3 can be expressed as parallel type (or antiparallel type). The structural type 2 can be expressed as cross type because the partial nucleic acid structure 1 and the partial nucleic acid structure 2 are generally fixed in a crossed state.

The shape of the partial structure 1 and partial structure 2 is not particularly limited. Examples of the shape include rectangle, triangle, polygon, ellipse, fan, polyhedron, cone, column, ellipse, etc., or a combination of these shapes. In an embodiment of the present invention, the shape is preferably elongated (e.g., the longest diameter is, for example, 1.5 times or more, 2 times or more, or 4 times or more the shortest diameter (excluding the thickness)).

The size of the partial structure 1 and partial structure 2 is not particularly limited, and can be suitably adjusted depending on the shape, size, etc. of the test substance. The partial structure 1 and partial structure 2 are each preferably nano-sized (nano-structure). For example, the longest diameter (major diameter) of each of the partial structure 1 and partial structure 2 is 1000 nm or less. In an embodiment of the present invention, the major diameter of each of the partial structure 1 and partial structure 2 is, for example, 5 to 1000 nm, preferably 20 to 800 nm, more preferably 50 to 600 nm, and even more preferably 100 to 400 nm.

The partial nucleic acid structure 1 contains a protruding structure 1a containing a nucleic acid aptamer sequence, and the partial nucleic acid structure 2 contains a protruding structure 2a containing a sequence complementary to part or all of the nucleic acid aptamer sequence.

The nucleic acid aptamer sequence is not particularly limited as long as it is a nucleic acid sequence that can bind (preferably specifically bind) to a substance (recognition substance). The recognition substance is not particularly limited as long as it can bind to the nucleic acid aptamer, and examples include nucleic acids, proteins, peptides, low-molecular-weight compounds, physiologically active substances, metal ions, and the like. In an embodiment of the present invention, the recognition substance is preferably a biological material. When the recognition substance is a nucleic acid, the nucleic acid aptamer sequence contains a sequence complementary to the nucleic acid, which is a recognition substance. The nucleic acid aptamer sequence can be a nucleic acid aptamer sequence known for a substance, or a sequence obtained by screening using a known method (e.g., SELEX method). The number of bases in the nucleic acid aptamer sequence is not particularly limited, and is, for example, 5 to 500, preferably 10 to 250, and more preferably 20 to 100.

The sequence complementary to part or all of the nucleic acid aptamer sequence is not particularly limited, but is preferably a sequence complementary to part of the nucleic acid aptamer sequence (e.g., 70% or less, 60% or less, or 50% or less of the nucleic acid aptamer sequence).

The protruding structure 1a and the protruding structure 2a are not particularly limited as long as they contain a nucleic acid aptamer sequence or a sequence complementary to part or all of the nucleic acid aptamer sequence, and protrude in the partial nucleic acid structure 1 and partial nucleic acid structure 2 (e.g., not incorporated into the structure due to complementary pairing etc.). For example, the protruding structure 1a and protruding structure 2a are part of the staple nucleic acid. In this example, the staple nucleic acid contains a sequence complementary to the single-stranded circular nucleic acid, and a nucleic acid aptamer sequence or a sequence complementary to part or all of the nucleic acid aptamer sequence, and the latter forms a protruding structure consisting of a single-stranded nucleic acid without complementarily binding to the single-stranded circular nucleic acid.

The number of protruding structures 1a is not particularly limited, and is, for example, 1 to 30, 2 to 20, 3 to 10, or 2 to 7. The number of protruding structures 2a is not particularly limited, and is, for example, 1 to 30, 2 to 20, 3 to 10, or 2 to 7.

The partial nucleic acid structure 1 contains a protruding structure 1b containing a split domain 1 of an enzyme, and the partial nucleic acid structure 2 contains a protruding structure 2b containing a split domain 2 paired with the split domain 1.

The enzyme used in the present invention is not particularly limited as long as it is a nucleic acid or protein that itself has enzymatic activity or that can exhibit enzymatic activity by binding to other substances, such as cofactors, and as long as it can be split into two (i.e., even if split, it can reassociate and exhibit enzymatic activity). The enzyme is preferably a nucleic acid enzyme containing two or more G-quartets, a photoprotein, or the like. The G-quartet is a planar structure formed by four guanine bases through Hoogsteen hydrogen bonding. Two or more of these are stacked to form a higher-order structure known as a guanine quadruplex. The higher-order structure can exhibit peroxidase activity in the presence of heme. Examples of photoproteins include NanoLuc (Promega). NanoLuc is a luciferase with furimazine as a substrate, and its luminescence intensity is very strong, about 100 times the luminescence intensity of firefly luciferase. LgBiT (Promega) and SmBiT (Promega) are subunits of NanoLuc, and they are reconstituted as a luciferase by interacting with each other. LgBiT is a large subunit of approximately 18 kDa, and SmBIT is a small subunit of 11 amino acid residues. LgBiT and SmBiT have a dissociation constant of 190 μM and extremely low affinity, and they do not express luciferase activity when simply mixed (Andrew, D., (2016) ACS Chem. Biol. 11, 400-408).

The split domain 1 and the split domain 2 are obtained by dividing an enzyme into two. The split position is not particularly limited as long as these domains can reassociate and exhibit enzymatic activity. When the enzyme is a nucleic acid enzyme containing two or more G-quartets, the split domain 1 (split nucleic acid sequence 1) and split domain 2 (split nucleic acid sequence 2) are not particularly limited as long as they are nucleic acid sequences that can associate to form G-quartets in the presence of a monovalent cation (e.g., potassium ion or sodium ion). Examples of such nucleic acid sequences include nucleic acid sequences in which a structural unit consisting of two or more (e.g., 2 to 4, or 2 or 3) consecutive guanine bases and a structural unit consisting of one or more (e.g., 1 to 5, or 2 or 3) consecutive non-guanine bases are alternately arranged. Typical examples of such nucleic acid sequences include telomere element sequences. When the enzyme is a photoprotein such as NanoLuc, the split domain 1 (split peptide sequence 1) and split domain 2 (split peptide sequence 2) are not particularly limited as long as they are peptide sequences that can express luciferase activity under conditions that allow both domains to remain stable in close proximity. The split peptide sequences can be determined pursuant to or according to known information, depending on the type of photoprotein, the desired dissociation constant, etc.

The protruding structure 1b and the protruding structure 2b are not particularly limited as long as they contain a split domain 1 or a split domain 2, and protrude in the partial nucleic acid structure 1 and the partial nucleic acid structure 2 (e.g., not incorporated into the structure due to complementary pairing etc.). For example, the protruding structure 1b and the protruding structure 2b are part of the staple nucleic acid. In this example, the staple nucleic acid contains a sequence complementary to the single-stranded circular nucleic acid, and the split domain 1 or split domain 2, and the latter forms a protruding structure consisting of a single-stranded nucleic acid without complementarily binding to the single-stranded circular nucleic acid.

The number of protruding structures 1b is not particularly limited, and is, for example, 1 to 30, 2 to 20, 3 to 10, or 2 to 7. The number of protruding structures 2b is not particularly limited, and is, for example, 1 to 30, 2 to 20, 3 to 10, or 2 to 7.

The arrangement of the protruding structure 1a and protruding structure 2a is not particularly limited as long as the protruding structure 1a and protruding structure 2a are arranged to face each other and bind together based on complementary base pairing in the absence of a recognition substance for the nucleic acid aptamer sequence. For example, in the case of taking the structural type 1, the protruding structure 1a and the protruding structure 2a are arranged to face each other. When the protruding structure 1a and the protruding structure 2a face each other, it is preferable that the protruding structure 1b and the protruding structure 2b do not face each other.

The arrangement of the protruding structure 1b and protruding structure 2b is not particularly limited as long as the protruding structure 1b and the protruding structure 2b are arranged to face each other to form a nucleic acid enzyme in the presence of a recognition substance for the nucleic acid aptamer sequence (when the nucleic acid enzyme contains two or more G-quartets, further in the presence of a monovalent cation). For example, in the case of taking the structural type 3, the protruding structure 1b and the protruding structure 2b are arranged to face each other. When the protruding structure 1b and the protruding structure 2b face each other, it is preferable that the protruding structure 1a and the protruding structure 2a do not face each other.

The protruding structure 1a, protruding structure 2a, protruding structure 1b, and protruding structure 2b are preferably arranged near the Holliday junction, which is the fulcrum. For example, these protruding structures are preferably arranged, for example, within 300 bases, within 200 bases, or within 150 bases, from the boundary between the two arms that form the Holliday junction in the partial nucleic acid structure 1 or partial nucleic acid structure 2. This makes it possible to keep the target structure more stable in the absence or presence of a test substance, and thus further increase the detection sensitivity of the test substance.

The nucleic acid structure of the present invention can be produced by or according to a known method. For example, the nucleic acid structure of the present invention can be produced by heating a solution containing a single-stranded circular nucleic acid and a staple nucleic acid containing a sequence complementary to the single-stranded circular nucleic acid to a high temperature (e.g., 80° C. or higher, 85° C. or higher, or 90° C. or higher) where base pairing hardly occurs, followed by gradual cooling (so that the temperature drops by 1° C., for example, for 15 seconds to 5 minutes, 30 seconds to 3 minutes, or 45 seconds to 2 minutes).

### 3. Use

As an example is shown in FIG. 13, in the nucleic acid structure of the present invention, in the absence of a test substance, the protruding structure 1a and the protruding structure 2a face each other and bind together based on complementary base pairing, and the structural type 1 (or structural type 3) becomes dominant. On the other hand, in the presence of a test substance, the bond between the protruding structure 1a and the protruding structure 2a is dissociated by the binding of the protruding structure 1a to the test substance. Along with this, the protruding structure 1b and the protruding structure 2b face each other to form a nucleic acid enzyme, and the structural type 3 (or structural type 1) becomes dominant. Accordingly, the test substance can be detected by detecting the activity of the nucleic acid enzyme. Therefore, the nucleic acid structure of the present invention can be used for detecting a test substance (e.g., as a constituent of a test substance detection composition).

From this point of view, the present invention, in an embodiment, relates to a test substance detection composition (the composition of the present invention) comprising the nucleic acid structure of the present invention, and a method for detecting a test substance, comprising bringing the nucleic acid structure of the present invention into contact with a sample that may contain the test substance (the detection method of the present invention). These are described below.

The test substance is not particularly limited as long as it can bind to a nucleic acid aptamer. Examples include nucleic acids, proteins, peptides, low-molecular-weight compounds, physiologically active substances, metal ions, and the like. In an embodiment of the present invention, the test substance is preferably a biological material.

When the test substance is a biological material, in an embodiment thereof, organisms containing the biological material can be detected. Examples of such organisms include viruses. Examples of viruses include enveloped viruses (viruses with envelopes), such as influenza viruses (e.g., type A and type B), rubella virus, Ebola virus, coronaviruses, measles virus, varicella-zoster virus, mumps virus, arbovirus, RS virus, SARS virus, hepatitis viruses (e.g., hepatitis B virus and hepatitis C virus), yellow fever virus, AIDS virus, rabies virus, hantavirus, dengue virus, Nipah virus, and Lyssa virus; non-enveloped viruses (viruses without envelopes), such as adenovirus, norovirus, rotavirus, human papillomavirus, poliovirus, enterovirus, coxsackievirus, human parvovirus, encephalomyocarditis virus, poliovirus, and rhinovirus; and the like. Particularly preferred among these are coronaviruses.

Coronaviruses belong to the subfamily Orthocoronavirinae. Examples of coronaviruses include alphacoronaviruses, betacoronaviruses, gammacoronaviruses, deltacoronaviruses, and the like. Preferred among these are betacoronaviruses. Examples of betacoronaviruses include SARS-associated coronavirus (SARSr-CoV), human coronavirus HKU1, MERS coronavirus, and the like. Preferred among these is SARSr-CoV. Examples of SARSr-CoV include SARS-CoV-2, SARS-CoV-1, and the like. Preferred among these is SARS-CoV-2. The agent of the present invention can be preferably used particularly against SARS-CoV-2.

The composition of the present invention may contain other components, if necessary. Examples of other components include bases, carriers, solvents, dispersants, emulsifiers, buffers, stabilizers, excipients, binders, disintegrants, lubricants, thickeners, humectants, colorants, fragrances, chelating agents, and the like.

The composition of the present invention may constitute a kit. The kit may include instruments, reagents, etc. for use in the implementation of the test method of the present invention. Examples of instruments include test tubes, microtiter plates, agarose particles, purification columns, epoxy-coated glass slides, gold colloid-coated glass slides, microtubes, and the like. Examples of reagents include cofactors of the nucleic acid enzyme, monovalent cation-containing buffers, standard samples (positive control and negative control), and the like.

The sample that may contain a test substance used in the detection method of the present invention is not particularly limited. Examples include biological samples. Specific examples include body fluids, such as whole blood, serum, plasma, follicular fluid, menstrual blood, saliva, cerebrospinal fluid, synovial fluid, urine, interstitial fluid, sweat, tears, and saliva; samples derived from these body fluids (e.g., purified products), tissue pieces, samples derived from tissue pieces (e.g., crushed products and purified products), and the like.

Before, during, or after contact of the nucleic acid structure of the present invention with the sample, a monovalent cation can be added, if necessary. For example, when the nucleic acid enzyme contains two or more G-quartets, G-quartets are formed by the presence of a monovalent cation.

Before, during, or after contact of the nucleic acid structure of the present invention with the sample, a cofactor of the nucleic acid enzyme can be added, if necessary. For example, when the nucleic acid enzyme contains two or more G-quartets, peroxidase activity is exerted by the presence of heme, such as hemin.

In an embodiment of the present invention, contact is performed in a solution, but is not limited thereto. The concentration of the nucleic acid structure of the present invention in the solution is not particularly limited, and is, for example, 0.1 to 100 nM, preferably 0.5 to 20 nM, and more preferably 1.5 to 10 nM. When the solution contains a monovalent cation, the concentration thereof is, for example, 10 mM to 1 M, preferably 50 to 600 mM, and more preferably 100 to 300 mM.

The pH of the solution in which contact is performed is preferably 2 to 10, more preferably 3.5 to 7.5, and even more preferably 4.5 to 5.5, in terms of allowing detection in a shorter time.

After contact, the enzymatic activity of the nucleic acid enzyme can be detected to detect the test substance. The enzymatic activity can be detected by or according to a known method. In a preferred embodiment of the present invention, it is preferable to add a substrate that becomes colored due to the enzymatic activity of the nucleic acid enzyme. As a result, the presence or absence of color change (i.e., the presence or absence and amount of test substance) can be detected or measured by visual observation or simple absorbance measurement. When the enzymatic activity is peroxidase activity, examples of such substrates include TMB (3,3′,5,5′-tetramethylbenzidine), OPD (o-phenylenediamine dihydrochloride), ABTS (2,2′-azinobis [3-ethylbenzothiazoline-6-sulfonic acid]-diammonium salt), and the like.

### EXAMPLES

The present invention is described in detail below based on Examples; however, the present invention is not limited by these Examples.

### Reference Example 1: Preparation 1 of Nucleic Acid Structure

As single-stranded circular DNA, M13mp18 (SEQ ID NO: 273) was purchased and prepared from tilibit nanosystems. Further, staple DNAs (SEQ ID NOs: 1 to 236) constituting a nucleic acid structure by complementary binding to the single-stranded circular DNA were purchased and prepared from IDT. M13mp18 and each staple DNA were mixed in a solution containing 40 mM of Tris, 20 mM of acetic acid, 2 mM of EDTA, and 12.5 mM of magnesium acetate (1×TAE/Mg²⁺ solution) so that the final concentration of M13mp18 was 4 nM and the final concentration of each staple DNA was 20 nM. The mixed solution was heated to 90° C. by a PCR thermal cycler and then cooled to 25° C. at a rate of—1.0° C. per minute. Excess staple was removed using a centrifugal ultrafiltration filter (Amicon Ultra 0.5 mL-100 K, Millipore).

Atomic force microscope (AFM) observation was performed, and it was confirmed that a pincher- or plier-like nucleic acid structure was formed. The major diameter was around 200 nm.

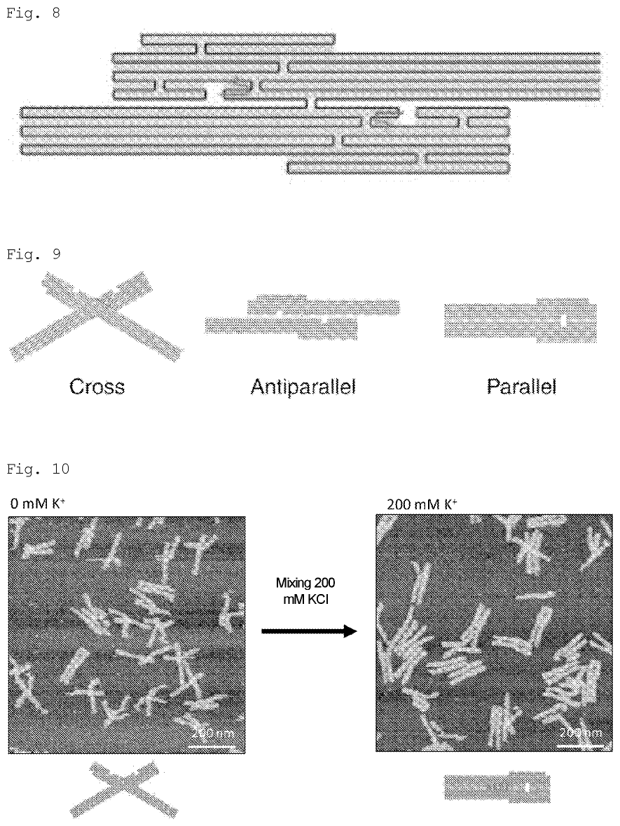

Tables 1 to 6 show the staple numbers, base sequences, and SEQ ID Nos of the staple DNAs used. FIG. 1 shows the overall view of the nucleic acid structure, and FIGS. 2 to 7 show diagrams obtained by dividing the overall view into six parts. Further, FIG. 8 shows a schematic diagram showing only single-stranded circular DNA in a linear form in the nucleic acid structure.

TABLE 1

| staple | | SEQ ID |
|---|---|---|
| No. | Sequence | No |
| 1s | CCTAATTTTTTTATCCTGAATCTTACCAACGCTATTIT | 1 |
| 2s | TTTTACGAGCGTCTGCAGCACCGTTTTT | 2 |
| 3s | CAAGTTTGAATGAAACCATCGATATTCCAGAG | 3 |
| 4s | AGCTACAAGCCAGTTACAAAATAAGGCCGGAA | 4 |
| 5s | ACGTCACCCCTTTAGCGTCAGACTACAACGCC | 5 |
| 6s | ATTATTTAAAGATTAGTTGCTATTTTGCACCC | 6 |
| 7s | TTTTCATCCCATTACCATTAGCAAACAGCCAT | 7 |
| 8s | TTGAAGCCTTAAATCTCCCAAT | 8 |
| 9s | CCAAATAAGAAACGATTTAGAGCCAGCA | 9 |
| 10 | TITTAATCAGTAGCATAGTTAGCGTTTT | 10 |
| 11 | TCGTCTTTTCCACAGACAGCCCTCGACAGAAT | 11 |
| 12 | TTTTTAACGATCTAAAGTTTTG | 12 |
| 13s | AAATCACCAGTAGCAGGCATTT | 13 |
| 14s | TGCCATCTGAGCCATTTGGGAATTTTTGTTTA | 14 |
| 15s | GCGTTTTAAATGAAAATAGCAGCCTCACCGTC | 15 |
| 16s | ACCGACTTTTTCATAATCAAAATCCTCATTTT | 16 |
| 17s | TAAAAACAATAGAAGGCTTATCCGGTATTCTAAGAACGCGAG | 17 |
| 18s | AGGTGAATTATTTACAGAGAGAATAACA | 18 |
| 19s | ACCGGAACGGAAATTATTCATTAA | 19 |
| 20s | AATCAGATGGGAAGCGCATTAGACGAAGGTAA | 20 |
| 21s | ATATTGACCGCCTCCCTCAGAGCCAGAACCGC | 21 |
| 22s | TAACTGAACATTACCGCGCCCAATAGCAAGCA | 22 |
| 23 | TGTATGGGGTCACCAGTACAAACTGTAGCGCG | 23 |
| 24 | TGTAGCATCCAGACGTTAGTAAATGAATTTTC | 24 |
| 25s | CAGAACCGAACCGATTGAGGGAGGGGGAGAAT | 25 |
| 26s | GTAGGAATCACCCTGAACAAAGTCCAAAAGGG | 26 |
| 27s | CGACATTCCCACCCTCAGAGCCACCCGTACTC | 27 |
| 28s | ATTGAGCGAAGCAAGCCGTTTTTATTTTCATC | 28 |
| 29s | GAGCCGCCTTACCAGCGCCAAAGAAGAGGGTA | 29 |
| 30s | TCGAGAACCTAATATCAGAGAGATAGAAAATT | 30 |
| 31s | CATATGGTACCAGAACCACCACCATGATATAA | 31 |
| 32s | AGAATTGAGTATTAAACCAAGTACCGCACTCA | 32 |
| 33 | TCGGTCATGTAACACTGAGTTTCATTTTGC | 33 |
| 34 | CCCATGTACCAGCCCCCTTATTAGCGTT | 34 |
| 35 | TAAACAACTITCAACAGTTTCAGCGG | 35 |
| 36s | GCCAGCATTTTTGTCACAATCAATAACCCACA | 36 |
| 37s | AAGAACGGGTTAAGCCCAATAATACCACGGAA | 37 |
| 38s | TAAGTTTATGACAGGAGGTTGAGGAGTACCAG | 38 |
| 39s | AAACAATGTCGGCTGTCTTTCCTTATCATTCC | 39 |
| 40s | ACGATTGGAAGAAACGCAAAGACAAGAGCAAG | 40 |

TABLE 2

| staple | | SEQ ID |
|---|---|---|
| No. | Sequence | No |
| 41 | AGTGAGAAGCAAGCCCAATAGGAA | 41 |
| 42s | ATCAATAAAAATAGCAATAGCTATAGGTGGCA | 42 |
| 43s | ACATATAACCTTGATATTCACAAAAGAGAAGG | 43 |
| 44s | AGCCCTITTAATTTACGAGCATGTAGAAACCA | 44 |
| 45s | TCCTCATTGAAAATACATACATAACTTACCGA | 45 |
| 46s | AAATAATATCCCATCCTTAAGAAAAGTAAGCATATGTTAG | 46 |
| 47s | CAAACGTAAAAGCCAGAATGGAAAACATGAAA | 47 |
| 48s | GAATCATAATAAGGCGTTAAATAAGA | 48 |
| 49s | AACAAAGTAAGTCCTGAACAAGAAAAACACCG | 49 |
| 50s | TCTGAATTACTCCTTATTACGCAGGATAGCCG | 50 |
| 51s | TGTGATAAATTACTAGAAAAAGCCTTTATCAA | 51 |
| 52 | AGCCACCACCACCGGAACCAGAGCCACC | 52 |
| 53 | CAGGGATATAGAAAGGAACAACTAAAGGAATTGC | 53 |
| 54 | GAATAATAATTTTTTCGAACCGCCACCCTCAG | 54 |
| 55s | CAATAGATTACCAGAAGGAAACCGAACTGGCA | 55 |
| 56s | TGATTAAGTACCGTTCCAGTAAGCCCCCTGCC | 56 |
| 57 | GCTCCAAATAGTACCGCCACCCTCGCCACCCT | 57 |
| 58 | CACCCTCAACGTTGAAAATCTCCAAAAAAAAG | 58 |
| 59s | ATCATATGTTTAATGGTTTGAAATACCGACCG | 59 |
| 60s | CAATAATAATGCAGAACGCGCCTGTGTTTAGT | 60 |
| 61 | ATCAGCTTCGGAATAGGTGTATCACACCCTCA | 61 |
| 62 | AGGAGGTTAGGAGCCTTTAATTGTATCGGTTT | 62 |
| 63s | TGGCTTTTACGGAATACCCAAAAGAGGAAACG | 63 |
| 64s | GACCTAAACGTTATACAAATTCTTACAACATG | 64 |
| 65 | AGCTTGATGTGCCGTCGAGAGGGTGAGCCGCC | 65 |
| 66 | GTATAGCCGCTTTCGAGGTGAATTTCTTAAAC | 66 |
| 67s | TTCAGCTAAGGTTTAACGTCAGATAATTGCGT | 67 |
| 68s | AAAGCCAAATATTTTAGTTAATTTCATCTTCT | 68 |
| 69 | AACAACCATAGCGGGGTTTTGCTCCAGGTCAG | 69 |
| 70 | GCGGATAAACCGATAGTTGCGCCGACAATGAC | 70 |
| 71s | AGTAACAGCAGACGACGACAATAAACCAGTAT | 71 |
| 72s | TTTCAAATCGCTCAACAGTAGGGCGTAAAGTA | 72 |
| 73 | CGGTCGCTAGGCTGAGACTCCTCACAAATAAA | 73 |
| 74 | ATTAGGATTCGCCCACGCATAACCGATATATT | 74 |
| 75s | ATTCTGTCTACCTITTACATCGGGAAAATTAT | 75 |
| 76s | GAATCGCCAAGACAAAGAACGCGAGAAAACTT | 76 |
| 77$ | TATTTCGGAACCTATTATTCTGAAGCGCAGTC | 77 |
| 78s | GTATTAAGGAGGCTTGCAGGGAGT | 78 |
| 79s | TAACGGATAAAGTACCGACAAAAGTTAATTGA | 79 |
| 80s | CCAATCGCATATTTAACAACGCCAAGTAATAA | 80 |

TABLE 3

| staple | | SEQ ID |
|---|---|---|
| No. | Sequence | No |
| 81c | TTGAATGGTATAAACAGTTAATGCGTCATACA | 81 |
| 82s | TAAAGGCCGCTTTTGCGAATACGTGGCACAGACAATATTT | 82 |
| 83w | AGATTTTCGATGATACAGGAGTGTTGAGTAAC | 83 |
| 84w | TAAGTTTTAAAACAGAAATAAAGAGAATATAC | 84 |
| 85c | GATAGCCCAACGGGGTCAGTGCCTACTGGTAA | 85 |
| 86c | AGTGCCCGCTATTAGTCTTTAATGCGCGAACT | 86 |
| 87w | TTGCACGTGGTGAGGCGGTCAGTACAGAAGAT | 87 |
| 88w | GCCTGCAATAGAACCTACCATATCAGAAACAA | 88 |
| 89w | CGAACCACCAGTTAACACC | 89 |
| 90w | AAAACAGATAAAACATCGC | 90 |
| 91w | CATTAAAAATACCGAA | 91 |
| 92w | GGAAGGGTCAGTGCCACGCTGAGA | 92 |
| 93w | CAAATGAAGTTTGGATTATACTTCAATACCAA | 93 |
| 94w | GCAAATCAACAGTTGAGCCAGCAG | 94 |
| 95w | AAGGAATTGAGGAAGG | 95 |
| 96w | TCCTGATTAAATCTAAAGCATCACAATATCTG | 96 |
| 97w | ACCTCAAAGATGATGGCAATTCATTTATTCAT | 97 |
| 98w | CTAACAACTATCAAACCCTCAATCCTTGCTGA | 98 |
| 99w | GTCAGTTGTTATCTAAAATATCTTTAGGAGCA | 99 |
| 100w | GATTATCAACGTTATTAATTTTAATAAATCCT | 100 |
| 101w | GTAACATTGAGCGGAATTATCATCTGATGAAA | 101 |
| 102w | TAATACATATTCGACAACTCGTATAAGTTTGA | 102 |
| 103w | TTGCCCGATAATAGATTAGAGCCGTCAATAGA | 103 |
| 104w | ACCAGAAGATCATTTTGCGGAACATTTTTTAGACTT | 104 |
| 105s | AACAATTTAAGAAAACAAAATTAATTTTAAGAAACC | 105 |
| 106w | TACAAACATTGAGGATTTAGAAGTTTTT | 106 |
| 107s | CAAACATCCATTTGAATTACCTTTAACATAGC | 107 |
| 108w | AAACAGTACCTGAGCAAAAGAAGAATATTCCT | 108 |
| 109s | TTCAATTACATAAATCAATATATGCTATTAAT | 109 |
| 110w | TAACCTTGATCGCGCAGAGGCGAACAATATAA | 110 |
| 111s | GAGAATATTCGCCTGATTGCTTTGTGAATAAT | 111 |
| 112s | TTTAGGCACTATATGTAAATGCTGATGCAAAT | 112 |
| 113s | GTTACAAAGAGGCATTTTCGAGCCACATGTAA | 113 |
| 114s | TTATATAACTTCTGTAAATCGTCGTGAGTGAA | 114 |
| 115s | TAATTTTCTITTAACCTCCGGCTTAGGTTGGG | 115 |
| 116s | GACTACCTCCTTAGAATCCTTGAATTTAATGG | 116 |
| 117s | GATAGCTTTTTATCAAAATCATAGGTCTGAGA | 117 |
| 118s | GAGTCAATAGTGAAAGATTAAGACGCTGAGTTTTTTACATTT | 118 |
| 119s | ACCATAAATCAAAAGTTCAGAAAACGAGAATTTTAAAATGTT | 119 |
| 120s | TTTAAACAATCAGGTCTTTACCCTGACTATTA | 120 |

TABLE 4

| staple | | SEQ ID |
|---|---|---|
| No. | Sequence | No |
| 121s | TAGTCAGAATTCATTGAATCCCCCCGGAATCG | 121 |
| 122s | GAGTAGATAGCAAAGCGGATTGCATCAAAAAG | 122 |
| 123w | AGACCAGGAAAGAGGACAGATGAATTTT | 123 |
| 124w | AGTGAATATCAACGTAACAAAGCTTTTTCGGTGTAC | 124 |
| 125s | AAAAGAAGTTTTGCATAGCGAGAGGCTTTTTTTTGCTCATTC | 125 |
| 126w | CAACTTTGCGCATAGGCTGGCTGAATATTCAT | 126 |
| 127w | TACCCAAAAGGCTTGCCCTGACGAGACGATAA | 127 |
| 128s | AAACCAAACAGAGGGGGTAATAGT | 128 |
| 129w | AAGAGTAACAATCATAAGGGAACCGAACTGAC | 129 |
| 130w | AGAACGAGTCTTGACAAGAACCGGCCTTCATC | 130 |
| 131w | TAGACTGGCCCTCGTTTACCAGACGAAACACC | 131 |
| 132w | CAGACGGTACAAAGTACAACGGAGTTATACCA | 132 |
| 133w | AGCGCGAATAGTAAATTGGGCTTGAGCAACAC | 133 |
| 134s | TATCATAAATAGCGTCCAATACTGTCAAATGC | 134 |
| 135w | CATCGCCTATGTTACTTAGCCGGAACGAGGCG | 135 |
| 136w | TAATTTCACTTTGACCCCCAGCGAATTTGTAT | 136 |
| 137w | TCATAAATTACGAGGCATAGTAAGAGATGGTT | 137 |
| 138w | ATCCGCGACCTGCTCC | 138 |
| 139w | GATAAATTGTGTCGAAACACTAAA | 139 |
| 140w | ACACTCATACTTTAATCATTGTGATAACGCCA | 140 |
| 141s | AAAGGAATTTAGTTTGACCATTAGCTGCGAAC | 141 |
| 142w | ATGCGATTAAAGAGGCAAAAGAAT | 142 |
| 143s | CGCAAATGAACTAATGCAGATACAATTACCTT | 143 |
| 144w | AGACTTTTTCATGAGG | 144 |
| 145w | AAGTTTCCATTGCACCAAC | 145 |
| 146w | CTAAAACGTTAAGAACTGGCTCATTAGGAATA | 146 |
| 147s | CCACATTCGTCAATAACCTGTTTAGTTTCATT | 147 |
| 148w | AAAATACGTTGAGGACTAA | 148 |
| 149w | TCAGGACGTAATGCCACTACGAAGAAACGGGT | 149 |
| 150s | TTCATTTGTTCATCAGTTGAGATTTATACCAG | 150 |
| 151w | ATTAAAGATTGGGAAGAAAAATCTATTACAGG | 151 |
| 152s | TAGAAAGAGGGCGCGAGCTGAAAATTAAATAT | 152 |
| 153w | AAAACGAATCCAGTTTGGAACAAGCAAAGGGC | 153 |
| 154s | CAATTCTACTAACGGAACAACATTACGTTAAT | 154 |
| 155s | AGTGTTGTTGAGGGGACGACGACACGTGCATC | 155 |
| 156s | TGCCAGTTCTAATAGTAGTAGCATTTGCTGAA | 156 |
| 157c | CAGAGGCTACGTGGACTCCAACGTAGTCCACT | 157 |
| 158c | GAAAAACCTCGGAACGAGGGTAGCAACGGCTA | 158 |
| 159s | AATAAATCATGGGCGCATCGTAACGTATCGGC | 159 |
| 160s | CTCAGGAAAAAGAATAGCCCGAGAGCCCACTA | 160 |

TABLE 5

| staple | | SEQ ID |
|---|---|---|
| No. | Sequence | No |
| 161c | AGACAGCAGTCTATCAGGGCGATGTAGGGTTG | 161 |
| 162s | TGACCTGAAAGCGTAAGGGATCGTCACCCTCAGCAGCGAA | 162 |
| 163s | GAATTAGCAAAATTAATGACCGTAATGGGATAGCTTTCCG | 163 |
| 164s | ATAAATCAGATCGCACTCCAGCCAGGTCACGT | 164 |
| 165s | CGTGAACCATCACCCAAATCAAGTAATCCCTT | 165 |
| 166s | GTCGAGGTAGAGATAGAACCCTTC | 166 |
| 167s | GCACCGCTGTTCCGAAATCGGCAATTTTTGGG | 167 |
| 168s | CTAAATCGCGTCGGATTCTCCGTGAGGCAAAG | 168 |
| 169 | TGGCCAACGCCGTAAAGCACTAAAATCCTGTT | 169 |
| 170 | CTAAAGGGCGACCAGTAATAAAAGGGACATTC | 170 |
| 171s | CGCCATTCGCCCCAGCAGGCGAAATCGGAACC | 171 |
| 172s | TGATGGTGTCTGGTGCCGGAAACCGGAACAAA | 172 |
| 173 | CAGTCACAAGCCCCCGATTTAGAGCGGTCCAC | 173 |
| 174 | GGAAAGCCGATTATTTACATTGGCAGATTCAC | 174 |
| 175s | GGAAGGGCAGAGAGTTGCAGCAAGCTTGACGG | 175 |
| 176s | GCTGGTTTGCCATTCAGGCTGCGCTGAGCGAG | 176 |
| 177 | CTGAAATGGGCGAACGTGGCGAGATCACCGCC | 177 |
| 178 | GAAGAAAGACCTACATTTTGACGCTCAATCGT | 178 |
| 179s | TACGCCAGAACAGCTGATTGCCCTAAGGAAGG | 179 |
| 180s | TGGCCCTGGATCGGTGCGGGCCTCTTCCTGTA | 180 |
| 181 | ATGGAAATCGAAAGGAGCGGGCGCCACCAGTG | 181 |
| 182 | TGGCAAGTGCCATTGCAACAGGAAAAACGCTC | 182 |
| 183s | AGGCGATTGGGTGGTTTTTTTTTTAGGGCGC | 183 |
| 184s | AGACGGGCCTGGCGAAAGGGGGATGGAACGCC | 184 |
| 185 | TACCGCCAGTAGCGGTCACGCTGCTGCGTATT | 185 |
| 186 | ACCACACCTGCTGGTAATATCCAGAACAATAT | 186 |
| 187s | CCAGTCACGGGGGGAGAGGCGGTTGCGTAACC | 187 |
| 188s | GGGCGCCAAAGTTGGGTAACGCCATTTTGTTA | 188 |
| 189 | CTCAAACTATCGGCCTCGCCGCGCTTAATGCG | 189 |
| 190 | TATGGTTGTAACATCACTTGCCTGAGTAGAAGAA | 190 |
| 191 | CCGCTACAGGAGCTGCATTAATGAATCG | 191 |
| 192s | ATGCCTGCGGAAACCTGTCGTGCCGCGCGTAC | 192 |
| 193s | GCCAACGCGACGTTGTAAAACGAC | 193 |
| 194s | GGCCAGTGCCTAAATTGTAAACGTTAAT | 194 |
| 195s | ATTTTGTTTTCAAAAGGGTGAGAAAGGCCGGAGACAGTCAAA | 195 |
| 196s | GGTAAAGAAAAATTCGCATTAAATGGGTTTTC | 196 |
| 197s | AATCAGCTAATGCAATGCCTGAGTAATGTGTA | 197 |
| 198s | ATATTTTACATTTTTTAACCAATAGTGCTGCA | 198 |
| 199s | ATCAAAAAGATAAAAATTTTTAGAACCCTCAT | 199 |
| 200s | AACGCAAGTAATTCGCGTCTGGCCTTCGCTAT | 200 |

TABLE 6

| staple | | SEQ ID |
|---|---|---|
| No. | Sequence | No |
| 201s | GCCAGCTTTTTTGCGGGAGAAGCCTTTATTTC | 201 |
| 202s | TGTAATACTCATCAACATTAAATGAACTGTTG | 202 |
| 203s | TAACAACCGTTGTACCAAAAACATTATGACCC | 203 |
| 204 | ATTAGTAACTTTGACGAGCACGTA | 204 |
| 205 | TTAACCGTTGTAGCAATACTTCTTTG | 205 |
| 206 | AATTGCGTTCCTCGTTAGAATCAACGCAAA | 206 |
| 207 | TAACGTGCTTTGCGCTCACTGCCCGCTT | 207 |
| 208s | TCCAGTCGAGGTCGACTCTAGAGGCCAAAAAC | 208 |
| 209s | TCACCATCTGTATAAGCAAATATTAAGCTTGC | 209 |
| 210s | ACCGAGCTCGAATTCTCACATT | 210 |
| 211s | TTGATAATCAGAAAAGCCATCCCCGGGT | 211 |
| 212s | AGGAAGATAATATGATATTCAACCGTTCTAGCTG | 212 |
| 213s | GAGCTAACGTAATCATGGTCATAGCATGTCAA | 213 |
| 214s | ATAAATTAATGCCGGACCCCGG | 214 |
| 215s | TCATATGTAGAGGGTAGCTATTTTTGAGAGAT | 215 |
| 216s | TGTGTGAATAAAGCCTGGGGTGCCGGAGGCCG | 216 |
| 217s | CTACAAAGGTAATCGTAAAACTAGCTGTTTCC | 217 |
| 218s | ATAAAGTGATTGTTATCCGCTCACAGAGAATC | 218 |
| 219s | GATGAACGGCTATCAGGTCATTGCCTGAGAGTCTTTTT | 219 |
| 220s | TTTTGGAGCAAACAAATTCCACACTTTT | 220 |
| 221 | TGTCCATCGAGCGGGAGCTAAACATAATGAGT | 221 |
| 222 | ATTAAAGGGTGAGGCCACCGAGTAAAAGAGTC | 222 |
| 223s | CGGCGGATGCAATAAAGCCTCAGAGCATAAAG | 223 |
| 224s | TGGTGTAGATACAGGCAAGGCAAAGAGGTCAT | 224 |
| 225s | TTTTGCGGTTAATTGCTCCTTTTG | 225 |
| 226s | GAGTACCTATGGCTTAGAGCTTAATAACATCC | 226 |
| 227s | TATAATGCAAACTCCAACAGGTCAGGATTAGA | 227 |
| 228s | CCGGAAGCTGTAGCTCAACATGTTGGTGGCAT | 228 |
| 229s | GCAACTAAATTCGAGCTTCAAAGCGAACCAGA | 229 |
| 230s | GCGTTTTAAGTACGGTGTCTGGAAGCTATATT | 230 |
| 231s | CCATATAAGAAGCCCGAAAGACTTCAAATATC | 231 |
| 232s | ATTAAGAGCAGTTGATTCCCAATTATACATTT | 232 |
| 233 | TATAATCAGATTITAGACAGGAACCCGGAAGC | 233 |
| 234 | TITTAATCCTGAGAAGTGTTTT | 234 |
| 235 | TITTAACATACGAGGGTACGCCAGTTTT | 235 |
| 8s-2 | ACGTCAAAGCGAACCTCCCGACTTGCGGGAGGTT | 236 |

### Reference Example 2: Preparation 2 of Nucleic Acid Structure

A nucleic acid structure was prepared in the same manner as in Reference Example 1, except that some staple DNAs were changed to other staple DNAs. Table 7 shows the corresponding staple numbers of Reference Example 1, and the staple numbers, base sequences, and SEQ ID Nos of the staple DNAs used. The configuration of the obtained nucleic acid structure was the same as that of the nucleic acid structure of Reference Example 1, except that the partial structure 1 and the partial structure 2 each contained a protruding structure consisting of single-stranded DNA of the following telomere element sequence.

TABLE 7

| Corresponding | | | |
|---|---|---|---|
| staple No. of | | | SEQ ID |
| Ref. Ex. 1 | staple No. | Sequence | No |
| 65 | 65long | TCTTAAACAGCTTGATGTGCCGTCGAGAGGGTGAGCCGCC | 237 |
| 66 | 66shortTelo | GTATAGCCGCTTTCGAGGTGAATTTTTTTAGGGTTAGGGT | 238 |
| 69 | 69long | ACAATGACAACAACCATAGCGGGGTTTTGCTCCAGGTCAG | 239 |
| 70 | 70shortTelo | GCGGATAAACCGATAGTTGCGCCGTTTTTAGGGTTAGGGT | 240 |
| 73 | 73long | GATATATTCGGTCGCTAGGCTGAGACTCCTCACAAATAAA | 241 |
| 74 | 74shortTelo | ATTAGGATTCGCCCACGCATAACCTTTTTAGGGTTAGGGT | 242 |
| 78s | 78s-telo | GTATTAAGGAGGCTTGCAGGGAGTTTTTTAGGGTTAGGGT | 243 |
| 166s | 166s-telo | GTCGAGGTAGAGATAGAACCCTTCTTTTTAGGGTTAGGGT | 244 |
| 169 | 169long | GGACATTCTGGCCAACGCCGTAAAGCACTAAAATCCTGTT | 245 |
| 170 | 170shortTelo | CTAAAGGGCGACCAGTAATAAAAGTTTTTAGGGTTAGGGT | 246 |
| 173 | 173long | AGATTCACCAGTCACAAGCCCCCGATTTAGAGCGGTCCAC | 247 |
| 174 | 174shortTelo | GGAAAGCCGATTATTTACATTGGCTTTTTAGGGTTAGGGT | 248 |
| 177 | 177long | TCAATCGTCTGAAATGGGCGAACGTGGCGAGATCACCGCC | 249 |
| 178 | 178shortTelo | GAAGAAAGACCTACATTTTGACGCTTTTTAGGGTTAGGGT | 250 |

Staple DNA with a staple number labeled “Telo” contains a telomere element sequence (including two G triplet repeats). The telomere element sequence forms a protruding structure consisting of single-stranded DNA without complementary binding to M13mp18. Among the three structures based on the conformation of the Holliday junction (FIG. 9: provided that Cross is an intermediate structure that mediates structural changes between Antiparallel and Parallel), when taking Parallel, the protruding structure in the partial structure 1 and the protruding structure in the partial structure 2 are arranged so that these protruding structures face each other (i.e., staple DNA containing a telomere element sequence is arranged). The telomere element sequence in the partial structure 1 and the telomere element sequence in the partial structure 2 can be linked in the presence of a monovalent cation to form a G-quartet structure (nucleic acid enzyme: heme stacks can exhibit peroxidase (HRP) activity).

A KCl solution (1 M KCl was dissolved in 1×TAE/Mg²⁺ solution) was added, or not added, to a solution containing the prepared nucleic acid structure, followed by still standing for 5 minutes. AFM observation was then performed, and the presence of K⁺ was visualized as a structural difference of the nucleic acid structure. The solution containing the nucleic acid structure contained 3.2 nM of the nucleic acid structure and 0 mM of KCl or 200 mM of KCl. Further, each structure of the nucleic acid structure was counted from the acquired images, and its proportion was examined.

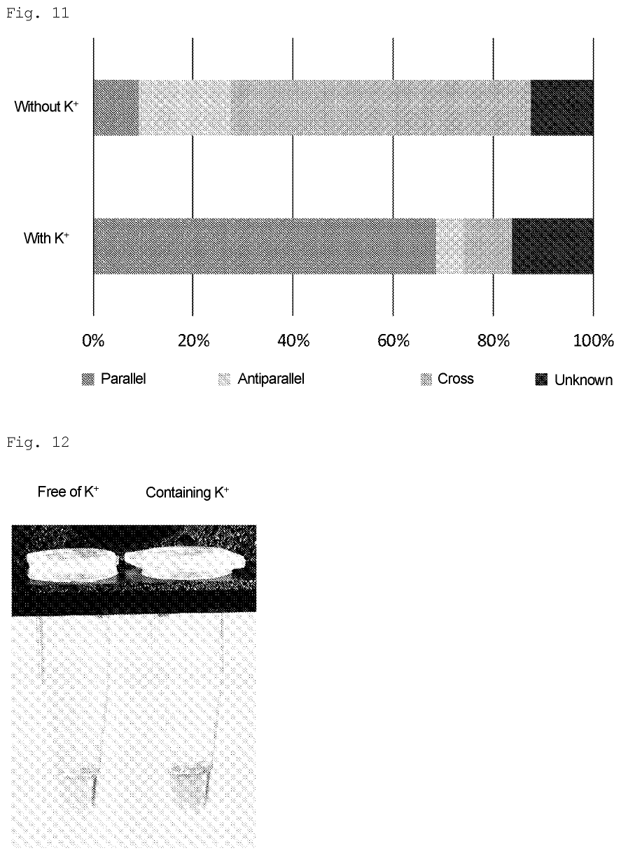

FIG. 10 shows AFM observation images. Further, FIG. 11 shows the proportion of each structure. A structural change to the parallel type due to the addition of K⁺ was confirmed.

### Reference Example 3: Detection 1 of Enzymatic Activity

A KCl solution (1 M KCl was dissolved in 1×TAE/Mg²⁺ solution) was added, or not added, to a solution containing the nucleic acid structure (Reference Example 2), followed by still standing for 5 minutes. Then, hemin (dissolved in DMSO at 100 μM) was added, followed by still standing for 1 hour. The solution containing the nucleic acid structure contained 3.2 nM of the nucleic acid structure, 4.0 μM of hemin, and 0 mM of KCl or 200 mM of KCl. Then, an equivalent amount of 1-Step Ultra TMB-ELISA (catalog number: 34028, Thermo Scientific) was added, and the presence of K⁺ was visualized as a color.

The results are shown in FIG. 12. A strong blue color was developed when a large amount of parallel nucleic acid structure was contained (K⁺ addition). This revealed that enzymatic activity can be exhibited by forming a G-quartet structure in the parallel nucleic acid structure.

### Example 1: Preparation 3 of Nucleic Acid Structure

A nucleic acid structure was prepared in the same manner as in Reference Example 1, except that some staple DNAs were changed to other staple DNAs. Tables 8 and 9 show the corresponding staple numbers of Reference Example 1, and the staple numbers, base sequences, and SEQ ID Nos of the staple DNAs used. The configuration of the obtained nucleic acid structure was the same as that of the nucleic acid structure of Reference Example 1, except that the partial structure 1 and the partial structure 2 each contained a protruding structure consisting of single-stranded DNA of a telomere element sequence, the partial nucleic acid structure 1 contained a protruding structure containing a nucleic acid aptamer sequence (complementary sequence of miR-20 (Mature sequence hsa-miR-20a-5p: SEQ ID NO: 274)), and the partial nucleic acid structure 2 contained a sequence complementary to part of the nucleic acid aptamer sequence.

TABLE 8

| Corresponding | | | |
|---|---|---|---|
| staple No. of | | | SEQ ID |
| Ref. Ex. 1 | staple No. | Sequence | No |
| 65 | 65long | TCTTAAACAGCTTGATGTGCCGTCGAGAGGGTGAGCCGCC | 251 |
| 66 | 66shortTelo | GTATAGCCGCTTTCGAGGTGAATTTTTTTAGGGTTAGGGT | 252 |
| 69 | 69long | ACAATGACAACAACCATAGCGGGGTTTTGCTCCAGGTCAG | 253 |
| 70 | 70shortTelo | GCGGATAAACCGATAGTTGCGCCGTTTTTAGGGTTAGGGT | 254 |
| 73 | 73LongCompM20 | GTGCAGGTAGTTTTGATATATTCGGTCGCTAGGCTGAGAC | 255 |
| | | TCCTCACAAATAAA | |
| 74 | 74shortTelo | ATTAGGATTCGCCCACGCATAACCTTTTTAGGGTTAGGGT | 256 |
| 78s | 78s-telo | GTATTAAGGAGGCTTGCAGGGAGTTTTTTAGGGTTAGGGT | 257 |
| 166s | 166s-telo | GTCGAGGTAGAGATAGAACCCTTCTTTTTAGGGTTAGGGT | 258 |
| 169 | 169LongCompM20 | GTGCAGGTAGTTTTGGAACATTTCTGGCCAACGCCGTAAA | 259 |
| | | GCACTAAAATCCTGTT | |
| 170 | 170shortTelo | CTAAAGGGCGACCAGTAATAAAAGTTTTTAGGGTTAGGGT | 260 |
| 173 | 173long | AGATTCACCAGTCACAAGCCCCCGATTTAGAGCGGTCCAC | 261 |
| 174 | 174shortTelo | GGAAAGCCGATTATTTACATTGGCTTTTTAGGGTTAGGGT | 262 |
| 177 | 177long | TCAATCGTCTGAAATGGGCGAACGTGGCGAGATCACCGCC | 263 |
| 178 | 178shortTelo | GAAGAAAGACCTACATTTTGACGCTTTTTAGGGTTAGGGT | 264 |

TABLE 9

| Corresponding | | | |
|---|---|---|---|
| staple No. of | | | SEQ ID |
| Ref. Ex. 1 | staple No. | Sequence | No |
| 81c | 81cLong | CAATATTTTTGAATGGTATAAACAGTTAATGCGTCATACA | 265 |
| 82s | 82sBothLongM20 | GTGCAGGTAGTTTTTAAAGGCCGCTTTTGCGAATACGTGG | 266 |
| | | CACAGATTTTCTACCTGCACTATAAGCACTTTA | |
| 85c | 85cLong | CGCGAACTGATAGCCCAACGGGGTCAGTGCCTACTGGTAA | 267 |
| 86c | 86cShortMainM20 | AGTGCCCGCTATTAGTCTTTAATGTTTTCTACCTGCACTAT | 268 |
| | | AAGCACTTTA | |
| 157c | 157cLong | AAACGGCTACAGAGGCTACGTGGACTCCAACGTAGTCCACT | 269 |
| 158c | 158cShortMainM20 | GAAAAACCTCGGAACGAGGGTAGCTTTTCTACCTGCACTAT | 270 |
| | | AAGCACTTTA | |
| 161c | 161cLong | GCAGCGAAAGACAGCAGTCTATCAGGGCGATGTAGGGTTG | 271 |
| 162s | 162sBothLongM20 | GTGCAGGTAGTTTTTGACCTGAAAGCGTAAGGGATCGTCA | 272 |
| | | CCCTCATTTTCTACCTGCACTATAAGCACTTTA | |

Staple DNA with a staple number labeled “Telo” contains a telomere element sequence (including two G triplet repeats). The structure formed by the telomere element sequence, its position, etc. are the same as those in Reference Example 2. The telomere element sequence in the partial structure 1 and the telomere element sequence in the partial structure 2 can be linked in the presence of miR-20 and a monovalent cation to form a G-quartet structure (nucleic acid enzyme: heme stacks can exhibit peroxidase (HRP) activity).

Staple DNA with a staple number labeled “M20” contains the complementary sequence of miR-20 or a sequence complementary to part of the sequence. These sequences form a protruding structure consisting of single-stranded DNA without complementary binding to M13mp18. Among the three structures based on the conformation of the Holliday junction (FIG. 9: provided that Cross is an intermediate structure that mediates structural changes between Antiparallel and Parallel), when taking Antiparallel, the protruding structure in the partial structure 1 and the protruding structure in the partial structure 2 are arranged so that these protruding structures face each other (i.e., staple DNA containing the complementary sequence of miR-20 or a sequence complementary to part of the sequence is arranged). The complementary sequence of miR-20 in the partial structure 2 and the sequence complementary to part of the sequence in the partial structure 1 can be bonded based on complementary base pairing in the absence of miR-20.

That is, as shown in FIG. 13, the nucleic acid structure of this Example is fixed at the antiparallel type based on complementary binding in the absence of miR-20, whereas in the presence of miR-20, the complementary bond dissociates to induce a structural change to the cross type, and the presence of a monovalent cation induces a structure change to the parallel type based on the formation of a G-quartet structure, resulting in the exhibition of peroxidase activity.

A KCl solution (1 M KCl was dissolved in 1×TAE/Mg²⁺ solution) was added to a solution containing the prepared nucleic acid structure, followed by still standing for 5 minutes. Then, a test substance, miR20 (dissolved in RNase-free Water (catalog number: 9012, TakaRa) at 200 μM), was added, followed by still standing for 1 hour. AFM observation was then performed, and the presence of miR20 was visualized as a structural difference of the nucleic acid structure. The solution containing the nucleic acid structure contained 3.2 nM of the nucleic acid structure, 1 μM of miR20, and 200 mM of KCl. Further, each structure of the nucleic acid structure was counted from the acquired images, and its proportion was examined.

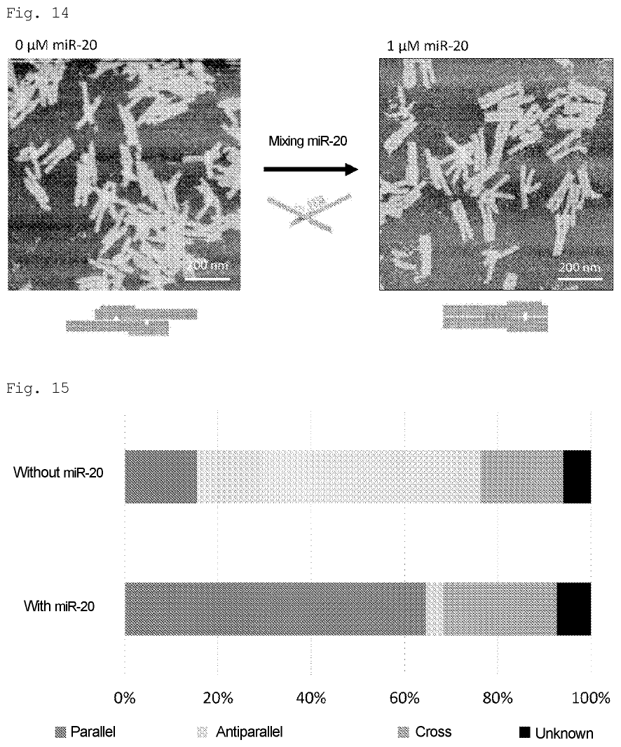

FIG. 14 shows AFM observation images. Further, FIG. 15 shows the proportion of each structure. The antiparallel type was the majority before the addition of miR-20, indicating that the addition of miR-20 induced a structural change from the antiparallel type to the parallel type.

### Example 2: Detection 2 of Enzymatic Activity

A KCl solution (1 M KCl was dissolved in 1×TAE/Mg²⁺ solution) was added to a solution containing the nucleic acid structure (Example 1), followed by still standing for 5 minutes. Then, a test substance, miR20 (dissolved in RNase-free Water (catalog number: 9012, TakaRa) at 200 μM), was added, followed by still standing for 1 hour. Further, hemin (dissolved in DMSO at 100 μM) was added, followed by still standing for 1 hour. The solution containing the nucleic acid structure contained 3.2 nM of the nucleic acid structure, 10 μM of miR20, 4.0 μM of hemin, and 200 mM of KCl. Then, an equivalent amount of 1-Step™ Ultra TMB-ELISA (catalog number: 34028, Thermo Scientific) was added, and the presence of miR20 was visualized as a color.

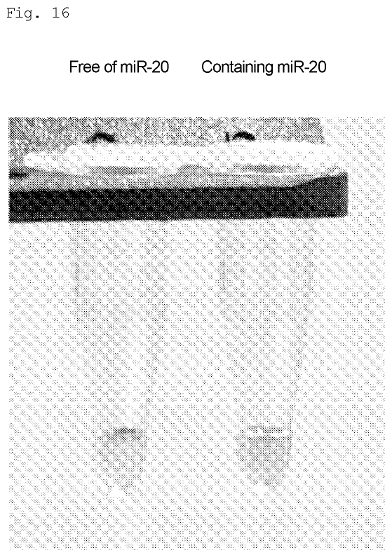

The results are shown in FIG. 16. A strong blue color was developed when miR-20 was contained. This revealed that miR-20 can be detected as a color change using the nucleic acid structure.

In conventional DNA nano-structures, split G-quartet sequences are highly stable, and it is difficult to maintain the split state; however, in the present invention, the pre-fixation on the opposite side (antiparallel type) successfully prevented the leakage of enzymatic activity. By using the technique of the present invention, the enzymatic activity can be controlled, for example, by varying the number of DNA strands attached to biomolecules. Unlike immunochromatography, the technique of the present invention is superior in that it does not require the fixation of antibodies or aptamers to underlying membranes or the like. In addition, the technique of the present invention, which can be composed only of nucleic acids, allows mass synthesis in a short time, cheaply and easily, with less lot differences compared to antibodies.

### Example 3: Preparation 4 of Nucleic Acid Structure

A nucleic acid structure was prepared in the same manner as in Reference Example 1, except that some staple DNAs were changed to other staple DNAs. Tables 10 and 11 show the corresponding staple numbers of Reference Example 1, and the staple numbers, base sequences, and SEQ ID Nos of the staple DNAs used. The configuration of the obtained nucleic acid structure was the same as that of the nucleic acid structure of Reference Example 1, except that the partial structure 1 and the partial structure 2 each contained a protruding structure consisting of single-stranded DNA of a telomere element sequence, the partial nucleic acid structure 1 contained a protruding structure containing a nucleic acid aptamer sequence (complementary sequence of R509 (SEQ ID NO: 297)), and the partial nucleic acid structure 2 contained a sequence complementary to part of the nucleic acid aptamer sequence. R509 is the complementary sequence of NIID_WH-1_F509 (National Institute of Infectious Diseases, Manual for the Detection of Pathogen 2019-nCOV Ver. 2.9.1).

TABLE 10

| Corresponding | | | |
|---|---|---|---|
| staple No. of | | | SEQ ID |
| Ref. Ex. 1 | staple No. | Sequence | No |
| 65 | 65long | TCTTAAACAGCTTGATGTGCCGTCGAGAGGGTGAGCCGCC | 275 |
| 66 | 66shortTelo | GTATAGCCGCTTTCGAGGTGAATTTTTTTAGGGTTAGGGT | 276 |
| 69 | 69long | ACAATGACAACAACCATAGCGGGGTTTTGCTCCAGGTCAG | 277 |
| 70 | 70shortTelo | GCGGATAAACCGATAGTTGCGCCGTTTTTAGGGTTAGGGT | 278 |
| 73 | 73LongCompCF509 | CAGTTCGAGTTTTGATATATTCGGTCGCTAGGCTGAGACTCCT | 279 |
| | | CACAAATAAA | |
| 74 | 74shortTelo | ATTAGGATTCGCCCACGCATAACCTTTTTAGGGTTAGGGT | 280 |
| 78s | 78s-telo | GTATTAAGGAGGCTTGCAGGGAGTTTTTTAGGGTTAGGGT | 281 |
| 166s | 166s-telo | GTCGAGGTAGAGATAGAACCCTTCTTTTTAGGGTTAGGGT | 282 |
| 169 | 169LongCompCF509 | CAGTTCGAGTTTTGGAACATTTCTGGCCAACGCCGTAAAGCAC | 283 |
| | | TAAAATCCTGTT | |
| 170 | 170shortTelo | CTAAAGGGCGACCAGTAATAAAAGTTTTTAGGGTTAGGGT | 284 |
| 173 | 173long | AGATTCACCAGTCACAAGCCCCCGATTTAGAGCGGTCCAC | 285 |
| 174 | 174shortTelo | GGAAAGCCGATTATTTACATTGGCTTTTTAGGGTTAGGGT | 286 |
| 177 | 177long | TCAATCGTCTGAAATGGGCGAACGTGGCGAGATCACCGCC | 287 |
| 178 | 178shortTelo | GAAGAAAGACCTACATTTTGACGCTTTTTAGGGTTAGGGT | 288 |

TABLE 11

| Corresponding | | | |
|---|---|---|---|
| staple No. of | | | SEQ ID |
| Ref. Ex. 1 | staple No. | Sequence | No |
| 81c | 81cLong | CAATATTTTTGAATGGTATAAACAGTTAATGCGTCATACA | 289 |
| 82s | 82sBothLongCF509 | CAGTTCGAGTTTTTAAAGGCCGCTTTTGCGAATACGTGGCACA | 290 |
| | | GATTTTCTCGAACTGCACCTCATGG | |
| 85c | 85cLong | CGCGAACTGATAGCCCAACGGGGTCAGTGCCTACTGGTAA | 291 |
| 86c | 86cShortCF509 | AGTGCCCGCTATTAGTCTTTAATGTTTTCTCGAACTGCACCTCA | 292 |
| | | TGG | |
| 157c | 157cLong | AAACGGCTACAGAGGCTACGTGGACTCCAACGTAGTCCACT | 293 |
| 158c | 158cShortCF509 | GAAAAACCTCGGAACGAGGGTAGCTTTTCTCGAACTGCACCTC | 294 |
| | | ATGG | |
| 161c | 161cLong | GCAGCGAAAGACAGCAGTCTATCAGGGCGATGTAGGGTTG | 295 |
| 162s | 162sBothLongCF509 | CAGTTCGAGTTTTTGACCTGAAAGCGTAAGGGATCGTCACCCT | 296 |
| | | CATTTTCTCGAACTGCACCTCATGG | |

Staple DNA with a staple number labeled “Telo” contains a telomere element sequence (including two G triplet repeats). The structure formed by the telomere element sequence, its position, etc. are the same as those in Reference Example 2. The telomere element sequence in the partial structure 1 and the telomere element sequence in the partial structure 2 can be linked in the presence of R509 and a monovalent cation to form a G-quartet structure (nucleic acid enzyme: heme stacks can exhibit peroxidase (HRP) activity).

Staple DNA with a staple number labeled “CF509” contains the complementary sequence of R509 or a sequence complementary to part of the sequence. These sequences form a protruding structure consisting of single-stranded DNA without complementary binding to M13mp18. Among the three structures based on the conformation of the Holliday junction (FIG. 9: provided that Cross is an intermediate structure that mediates structural changes between Antiparallel and Parallel), when taking Antiparallel, the protruding structure in the partial structure 1 and the protruding structure in the partial structure 2 are arranged so that these protruding structures face each other (i.e., staple DNA containing the complementary sequence of R509 or a sequence complementary to part of the sequence is arranged). The complementary sequence of R509 in the partial structure 2 and the sequence complementary to part of the sequence in the partial structure 1 can be bonded based on complementary base pairing in the absence of R509.

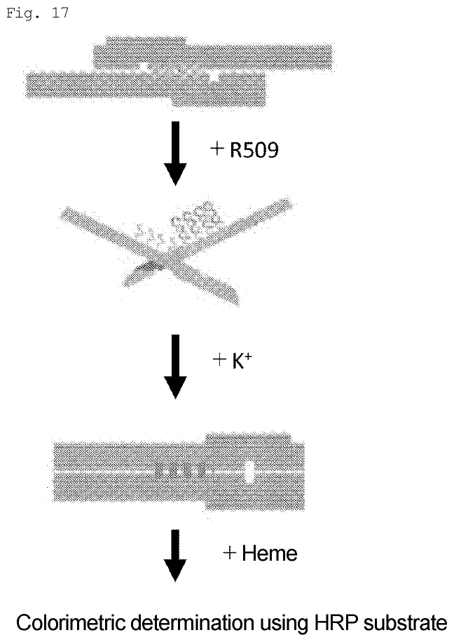

That is, as shown in FIG. 17, the nucleic acid structure of this Example is fixed at the antiparallel type based on complementary binding in the absence of R509, whereas in the presence of R509, the complementary bond dissociates to induce a structural change to the cross type, and the presence of a monovalent cation induces a structure change to the parallel type based on the formation of a G-quartet structure, resulting in the exhibition of peroxidase activity.

A KCl solution (1 M KCl was dissolved in 1×TAE/Mg²⁺ solution) was added to a solution containing the prepared nucleic acid structure, followed by still standing for 5 minutes. Then, a test substance, R509 (dissolved in RNase-free Water (catalog number: 9012, TakaRa) at 200 μM), or R29142 (SEQ ID NO: 344), which was not a test substance, was added, followed by still standing for 1 hour. AFM observation was then performed, and the presence of R509 was visualized as a structural difference of the nucleic acid structure. The solution containing the nucleic acid structure contained 3.2 nM of the nucleic acid structure, 1 μM of R509, and 200 mM of KCl. Further, each structure of the nucleic acid structure was counted from the acquired images, and its proportion was examined. R29142 is the complementary sequence of NIID_2019-nCOV_N_F2 (National Institute of Infectious Diseases, Manual for the Detection of Pathogen 2019-nCOV Ver. 2.9.1).

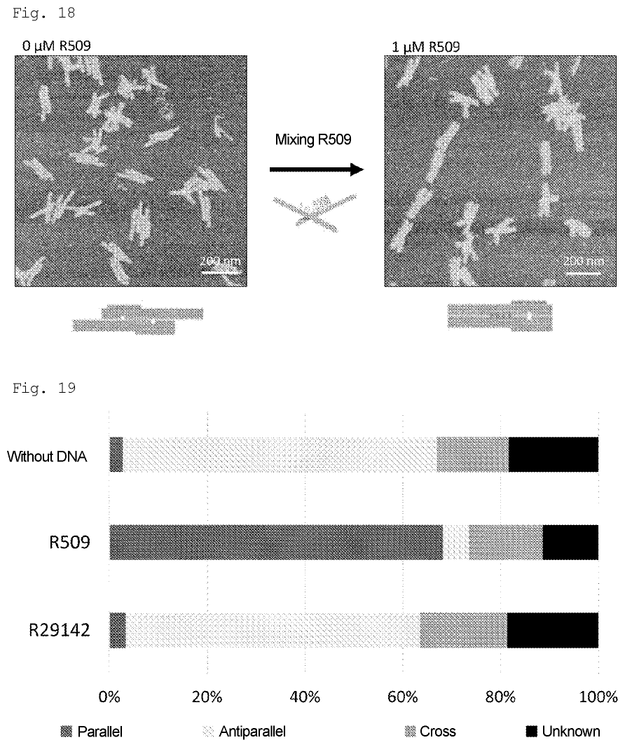

FIG. 18 shows AFM observation images. Further, FIG. 19 shows the proportion of each structure. The antiparallel type was the majority before the addition of R509, indicating that the addition of R509 induced a structural change from the antiparallel type to the parallel type. It was also indicated that the addition of R29142 did not induce a structural change from the antiparallel type to the parallel type.

### Example 4: Detection 3 of Enzymatic Activity

A KCl solution (1 M KCl was dissolved in 1×TAE/Mg²⁺ solution) was added to a solution containing the nucleic acid structure (Example 3), followed by still standing for 5 minutes. Then, a test substance, R509 (dissolved in RNase-free Water (catalog number: 9012, TaKaRa) at 200 μM), was added, followed by still standing for 1 hour. Further, hemin (dissolved in DMSO at 100 μM) was added, followed by still standing for 1 hour. The solution containing the nucleic acid structure contained 3.2 nM of the nucleic acid structure, 10 μM of R509, 4.0 μM of hemin, and 200 mM of KCl. Then, an equivalent amount of 1-Step™ Ultra TMB-ELISA (catalog number: 34028, Thermo Scientific) was added, and the presence of R509 was visualized as a color.

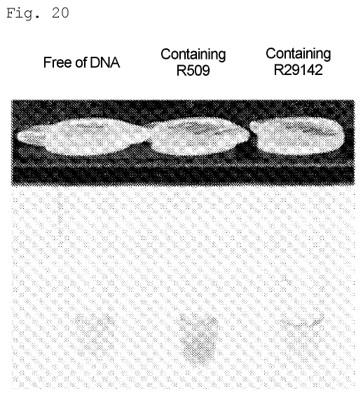

The results are shown in FIG. 20. A strong blue color was developed when R509 was contained. This revealed that R509 can be detected as a color change using the nucleic acid structure.

In conventional DNA nano-structures, split G-quartet sequences are highly stable, and it is difficult to maintain the split state; however, in the present invention, the pre-fixation on the opposite side (antiparallel type) successfully prevented the leakage of enzymatic activity. By using the technique of the present invention, the enzymatic activity can be controlled, for example, by varying the number of DNA strands attached to biomolecules. Unlike immunochromatography, the technique of the present invention is superior in that it does not require the fixation of antibodies or aptamers to underlying membranes or the like. In addition, the technique of the present invention, which can be composed only of nucleic acids, allows mass synthesis in a short time, cheaply and easily, with less lot differences compared to antibodies.

### Example 5: Preparation 5 of Nucleic Acid Structure

A nucleic acid structure was prepared in the same manner as in Reference Example 1, except that some staple DNAs were changed to other staple DNAs. Tables 12 and 13 show the corresponding staple numbers of Reference Example 1, and the staple numbers, base sequences, and SEQ ID Nos of the staple DNAs used. The configuration of the obtained nucleic acid structure was the same as that of the nucleic acid structure of Reference Example 1, except that the partial structure 1 and the partial structure 2 each contained a protruding structure consisting of single-stranded DNA of a telomere element sequence, the partial nucleic acid structure 1 contained a protruding structure containing a nucleic acid aptamer sequence (complementary sequence of N1 (SEQ ID NO: 320)), and the partial nucleic acid structure 2 contained a sequence complementary to part of the nucleic acid aptamer sequence. The complementary sequence of N1 is a sequence complementary to the nucleic acid sequence of N_Sarbeco_P1 (National Institute of Infectious Diseases, Manual for the Detection of Pathogen 2019-nCOV Ver. 2.9.1).

TABLE 12

| Corresponding | | | |
|---|---|---|---|
| staple No. of | | | SEQ ID |
| Ref. Ex. 1 | staple No. | Sequence | No |
| 65 | 65long | TCTTAAACAGCTTGATGTGCCGTCGAGAGGGTGAGCCGCC | 298 |
| 66 | 66shortTelo | GTATAGCCGCTTTCGAGGTGAATTTTTTTAGGGTTAGGGT | 299 |
| 69 | 69long | ACAATGACAACAACCATAGCGGGGTTTTGCTCCAGGTCAG | 300 |
| 70 | 70shortTelo | GCGGATAAACCGATAGTTGCGCCGTTTTTAGGGTTAGGGT | 301 |
| 73 | 73LongCompN1 | AACATTGCCATTTTGATATATTCGGTCGCTAGGCTGAGACTCCT | 302 |
| | | CACAAATAAA | |
| 74 | 74shortTelo | ATTAGGATTCGCCCACGCATAACCTTTTTAGGGTTAGGGT | 303 |
| 78s | 78s-telo | GTATTAAGGAGGCTTGCAGGGAGTTTTTTAGGGTTAGGGT | 304 |
| 166s | 166s-telo | GTCGAGGTAGAGATAGAACCCTTCTTTTTAGGGTTAGGGT | 305 |
| 169 | 169LongCompN1 | AACATTGCCATTTTGGACATTCTGGCCAACGCCGTAAAGCACTA | 306 |
| | | AAATCCTGTT | |
| 170 | 170shortTelo | CTAAAGGGCGACCAGTAATAAAAGTTTTTAGGGTTAGGGT | 307 |
| 173 | 173long | AGATTCACCAGTCACAAGCCCCCGATTTAGAGCGGTCCAC | 308 |
| 174 | 174shortTelo | GGAAAGCCGATTATTTACATTGGCTTTTTAGGGTTAGGGT | 309 |
| 177 | 177long | TCAATCGTCTGAAATGGGCGAACGTGGCGAGATCACCGCC | 310 |
| 178 | 178shortTelo | GAAGAAAGACCTACATTTTGACGCTTTTTAGGGTTAGGGT | 311 |

TABLE 13

| Corresponding | | | |
|---|---|---|---|
| staple No. of | | | SEQ ID |
| Ref. Ex. 1 | staple No. | Sequence | No |
| 81c | 81cLong | CAATATTTTTGAATGGTATAAACAGTTAATGCGTCATACA | 312 |
| 82s | 82sBothLongN1 | AACATTGCCATTTTTAAAGGCCGCTTTTGCGAATACGTGGCACA | 313 |
| | | GATTTTTGGCAATGTTGTTCCTTGAGGAAGT | |
| 85c | 85cLong | CGCGAACTGATAGCCCAACGGGGTCAGTGCCTACTGGTAA | 314 |
| 86c | 86cShortN1 | AGTGCCCGCTATTAGTCTTTAATGTTTTTGGCAATGTTGTTCCT | 315 |
| | | TGAGGAAGT | |
| 157c | 157cLong | AAACGGCTACAGAGGCTACGTGGACTCCAACGTAGTCCACT | 316 |
| 158c | 158cShortN1 | GAAAAACCTCGGAACGAGGGTAGCTTTTTGGCAATGTTGTTCC | 317 |
| | | TTGAGGAAGT | |
| 161c | 161cLong | GCAGCGAAAGACAGCAGTCTATCAGGGCGATGTAGGGTTG | 318 |
| 162s | 162sBothLongN1 | AACATTGCCATTTTTGACCTGAAAGCGTAAGGGATCGTCACCC | 319 |
| | | TCATTTTTGGCAATGTTGTTCCTTGAGGAAGT | |

Staple DNA with a staple number labeled “Telo” contains a telomere element sequence (including two G triple repeats). The structure formed by the telomere element sequence, its position, etc. are the same as those in Reference Example 2. The telomere element sequence in the partial structure 1 and the telomere element sequence in the partial structure 2 can be linked in the presence of N1 and a monovalent cation to form a G-quartet structure (nucleic acid enzyme: heme stacks can exhibit peroxidase (HRP) activity).

Staple DNA with a staple number labeled “N1” contains the complementary sequence of N1 or a sequence complementary to part of the sequence. These sequences form a protruding structure consisting of single-stranded DNA without complementary binding to M13mp18. Among the three structures based on the conformation of the Holliday junction (FIG. 9: provided that Cross is an intermediate structure that mediates structural changes between Antiparallel and Parallel), when taking Antiparallel, the protruding structure in the partial structure 1 and the protruding structure in the partial structure 2 are arranged so that these protruding structures face each other (i.e., staple DNA containing the complementary sequence of N1 or a sequence complementary to part of the sequence is arranged). The complementary sequence of N1 in the partial structure 2 and the sequence complementary to part of the sequence in the partial structure 1 can be bonded based on complementary base pairing in the absence of N1.

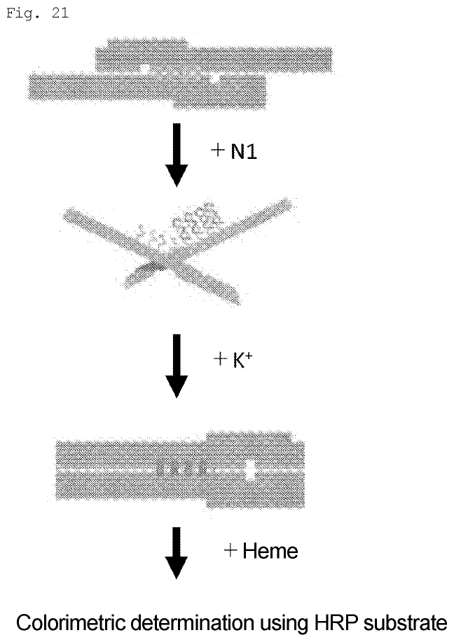

That is, as shown in FIG. 21, the nucleic acid structure of this Example is fixed at the antiparallel type based on complementary binding in the absence of N1, whereas in the presence of N1, the complementary bond dissociates to induce a structural change to the cross type, and the presence of a monovalent cation induces a structure change to the parallel type based on the formation of a G-quartet structure, resulting in the exhibition of peroxidase activity.

A KCl solution (1 M KCl was dissolved in 1×TAE/Mg²⁺ test substance, N1 (dissolved in RNase-free Water (catalog number: 9012, TakaRa) at 200 μM), was added, followed by still standing for 1 hour. AFM observation was then performed, and the presence of N1 was visualized as a structural difference of the nucleic acid structure. The solution containing the nucleic acid structure contained 3.2 nM of the nucleic acid structure, 1.0 μM of N1, and 200 mM of KCl. Further, each structure of the nucleic acid structure was counted from the acquired images, and its proportion was examined.

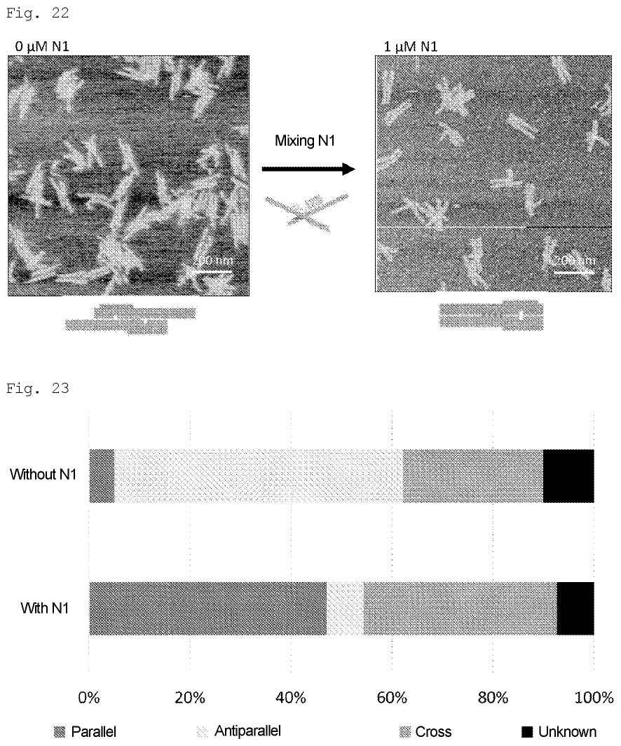

FIG. 22 shows AFM observation images. Further, FIG. 23 shows the proportion of each structure. The antiparallel type was the majority before the addition of N1, indicating that the addition of N1 induced a structural change from the antiparallel type to the parallel type.

### Example 6: Detection 4 of Enzymatic Activity

A KCl solution (1 M KCl was dissolved in 1×TAE/Mg²⁺ solution) was added to a solution containing the nucleic acid structure (Example 5), followed by still standing for 5 minutes. Then, a test substance, N1 (dissolved in RNase-free Water (catalog number: 9012, TakaRa) at 200 μM), was added, followed by still standing for 15 minutes. Further, hemin (dissolved in DMSO at 100 μM) was added, followed by still standing for 30 minutes. The solution containing the nucleic acid structure contained 7.5 nM of the nucleic acid structure, 2.0 μM of N1, 5.0 μM of hemin, 0.0004% (w/v) of Triton X-100, and 200 mM of KCl. Then, an equivalent amount of 1-Step™ Ultra TMB-ELISA (catalog number: 34028, Thermo Scientific, pH: 4.9) mixed with 1×TAE/Mg²⁺ and 200 mM of KCl was added (pH of the solution after mixing: 5.5), and 10 minutes later, the presence of N1 was visualized as a color.

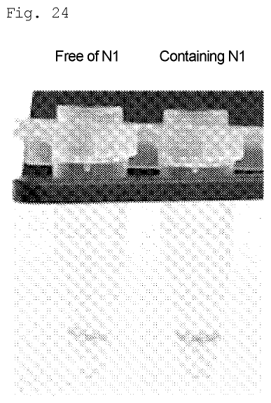

The results are shown in FIG. 24. A strong blue color was developed when N1 was contained. This revealed that N1 can be detected as a color change using the nucleic acid structure.

In conventional DNA nano-structures, split G-quartet sequences are highly stable, and it is difficult to maintain the split state; however, in the present invention, the pre-fixation on the opposite side (antiparallel type) successfully prevented the leakage of enzymatic activity. By using the technique of the present invention, the enzymatic activity can be controlled, for example, by varying the number of DNA strands attached to biomolecules. Unlike immunochromatography, the technique of the present invention is superior in that it does not require the fixation of antibodies or aptamers to underlying membranes or the like. In addition, the technique of the present invention, which can be composed only of nucleic acids, allows mass synthesis in a short time, cheaply and easily, with less lot differences compared to antibodies.

### Example 7: Preparation 6 of Nucleic Acid Structure

A nucleic acid structure was prepared in the same manner as in Reference Example 1, except that some staple DNAs were changed to other staple DNAs. Tables 14 and 15 show the corresponding staple numbers of Reference Example 1, and the staple numbers, base sequences, and SEQ ID Nos of the staple DNAs used. The configuration of the obtained nucleic acid structure was the same as that of the nucleic acid structure of Reference Example 1, except that the partial structure 1 and the partial structure 2 each contained a protruding structure consisting of single-stranded DNA of a telomere element sequence, the partial nucleic acid structure 1 contained a protruding structure containing a nucleic acid aptamer sequence (HD1 (thrombin aptamer, Bock, L. et al., (1992) Nature 355, 564-566, Munzar J. D. et al., (2018) Nat Commun. doi: 10.1038/s41467-017-02556-3): SEQ ID NO: 343), and the partial nucleic acid structure 2 contained a sequence complementary to part of the nucleic acid aptamer sequence.

TABLE 14

| Corresponding | | | |
|---|---|---|---|
| staple No. of | | | SEQ ID |
| Ref. Ex. 1 | staple No. | Sequence | No |
| 65 | 65long | TCTTAAACAGCTTGATGTGCCGTCGAGAGGGTGAGCCGCC | 321 |
| 66 | 66shortTelo | GTATAGCCGCTTTCGAGGTGAATTTTTTTAGGGTTAGGGT | 322 |
| 69 | 69long | ACAATGACAACAACCATAGCGGGGTTTTGCTCCAGGTCAG | 323 |
| 70 | 70shortTelo | GCGGATAAACCGATAGTTGCGCCGTTTTTAGGGTTAGGGT | 324 |
| 73 | 73LongCompHD1T1 | ACACCAACCTTTTTGATATATTCGGTCGCTAGGCTGAGACTCCT | 325 |
| | | CACAAATAAA | |
| 74 | 74shortTelo | ATTAGGATTCGCCCACGCATAACCTTTTTAGGGTTAGGGT | 326 |
| 78s | 78s-telo | GTATTAAGGAGGCTTGCAGGGAGTTTTTTAGGGTTAGGGT | 327 |
| 166s | 166s-telo | GTCGAGGTAGAGATAGAACCCTTCTTTTTAGGGTTAGGGT | 328 |
| 169 | 169LongCompHD1T1 | ACACCAACCTTTTTGGACATTCTGGCCAACGCCGTAAAGCACTA | 329 |
| | | AAATCCTGTT | |
| 170 | 170shortTelo | CTAAAGGGCGACCAGTAATAAAAGTTTTTAGGGTTAGGGT | 330 |
| 173 | 173long | AGATTCACCAGTCACAAGCCCCCGATTTAGAGCGGTCCAC | 331 |
| 174 | 174shortTelo | GGAAAGCCGATTATTTACATTGGCTTTTTAGGGTTAGGGT | 332 |
| 177 | 177long | TCAATCGTCTGAAATGGGCGAACGTGGCGAGATCACCGCC | 333 |
| 178 | 178shortTelo | GAAGAAAGACCTACATTTTGACGCTTTTTAGGGTTAGGGT | 334 |

TABLE 15

| Corresponding | | | |
|---|---|---|---|
| staple No. of | | | SEQ ID |
| Ref. Ex. 1 | staple No. | Sequence | No |
| 81c | 81cLong | CAATATTTTTGAATGGTATAAACAGTTAATGCGTCATACA | 335 |
| 82s | 82sBothLongHD1T1 | ACACCAACCTTTTTTAAAGGCCGCTTTTGCGAATACGTGGCACA | 336 |
| | | GATTTGTGGTTGGTGTGGTTGGTCATA | |
| 85c | 85cLong | CGCGAACTGATAGCCCAACGGGGTCAGTGCCTACTGGTAA | 337 |
| 86c | 86cShortHD1T1 | AGTGCCCGCTATTAGTCTTTAATGTTTTAGTCCGTGGTAGGGC | 338 |
| | | AGGTTGGGGTGACT | |
| 157c | 157cLong | AAACGGCTACAGAGGCTACGTGGACTCCAACGTAGTCCACT | 339 |
| 158c | 158cShortHD1T1 | GAAAAACCTCGGAACGAGGGTAGCTTTTAGTCCGTGGTAGGGC | 340 |
| | | AGGTTGGGGTGACT | |
| 161c | 161cLong | GCAGCGAAAGACAGCAGTCTATCAGGGCGATGTAGGGTTG | 341 |
| 162s | 162sBothLongHD1T1 | ACACCAACCTTTTTTGACCTGAAAGCGTAAGGGATCGTCACCC | 342 |
| | | TCATTTGTGGTTGGTGTGGTTGGTCATA | |

Staple DNA with a staple number labeled “Telo” contains a telomere element sequence (including two G triple repeats). The structure formed by the telomere element sequence, its position, etc. are the same as those in Reference Example 2. The telomere element sequence in the partial structure 1 and the telomere element sequence in the partial structure 2 can be linked in the presence of thrombin and a monovalent cation to form a G-quartet structure (nucleic acid enzyme: heme stacks can exhibit peroxidase (HRP) activity).

Staple DNA with a staple number labeled “HD1T1” contains the complementary sequence of HD1 or a sequence complementary to part of the sequence. These sequences form a protruding structure consisting of single-stranded DNA without complementary binding to M13mp18. Among the three structures based on the conformation of the Holliday junction (FIG. 9: provided that Cross is an intermediate structure that mediates structural changes between Antiparallel and Parallel), when taking Antiparallel, the protruding structure in the partial structure 1 and the protruding structure in the partial structure 2 are arranged so that these protruding structures face each other (i.e., staple DNA containing the complementary sequence of HD1T1 or a sequence complementary to part of the sequence is arranged). The complementary sequence of HD1T1 in the partial structure 2 and the sequence complementary to part of the sequence in the partial structure 1 can be bonded based on complementary base pairing in the absence of thrombin.

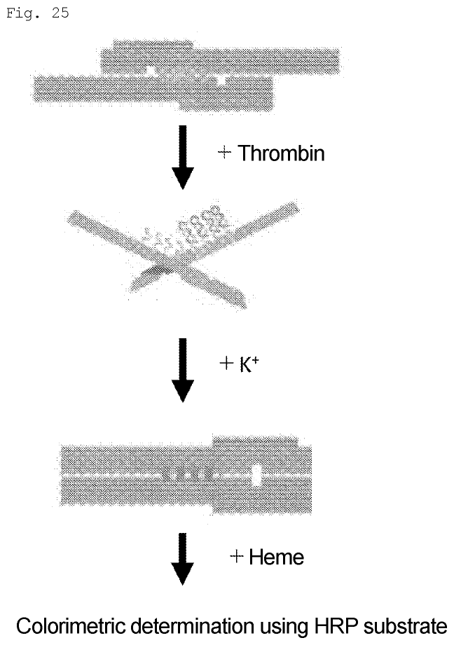

That is, as shown in FIG. 25, the nucleic acid structure of this Example is fixed at the antiparallel type based on complementary binding in the absence of thrombin, whereas in the presence of thrombin, the complementary bond dissociates to induce a structural change to the cross type, and the presence of a monovalent cation induces a structure change to the parallel type based on the formation of a G-quartet structure, resulting in the exhibition of peroxidase activity.

A KCl solution (1 M KCl was dissolved in 1×TAE/Mg²⁺ test substance, thrombin, was added, followed by still standing for 1 hour. AFM observation was then performed, and the presence of thrombin was visualized as a structural difference of the nucleic acid structure. The solution containing the nucleic acid structure contained 3.2 nM of the nucleic acid structure, 2.0 μM of thrombin, and 200 mM of KCl. Further, each structure of the nucleic acid structure was counted from the acquired images, and its proportion was examined.

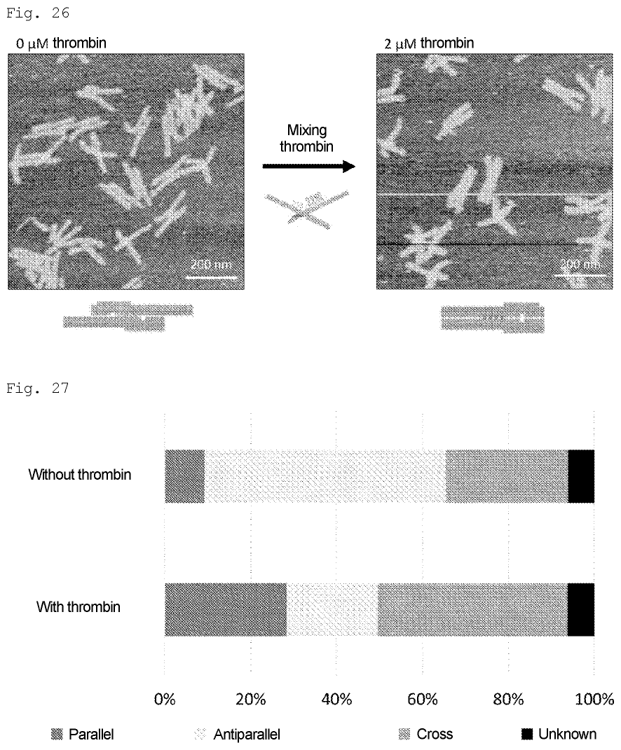

FIG. 26 shows AFM observation images. Further, FIG. 27 shows the proportion of each structure. The antiparallel type was the majority before the addition of thrombin, indicating that the addition of thrombin induced a structural change from the antiparallel type to the parallel type.

### Example 8: Detection 5 of Enzymatic Activity

A KCl solution (1 M KCl was dissolved in 1×TAE/Mg²⁺ solution) was added to a solution containing the nucleic acid structure (Example 7), followed by still standing for 5 minutes. Then, a test substance, thrombin, was added, followed by still standing for 15 minutes. Further, hemin (dissolved in DMSO at 100 μM) was added, followed by still standing for 30 minutes. The solution containing the nucleic acid structure contained 7.5 nM of the nucleic acid structure, 2.0 μM of thrombin, 5.0 μM of hemin, 0.0004% (w/v) of Triton X-100, and 200 mM of KCl. Then, an equivalent amount of 1-Step™ Ultra TMB-ELISA (catalog number: 34028, Thermo Scientific) was added, and the presence of thrombin was visualized as a color.

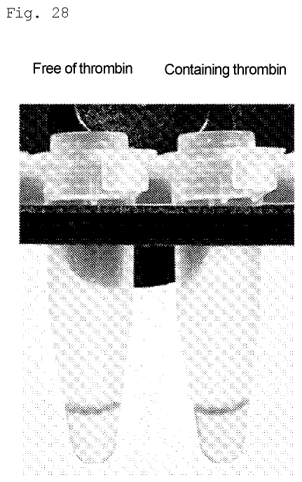

The results are shown in FIG. 28. A strong blue color was developed when thrombin was contained. This revealed that thrombin can be detected as a color change using the nucleic acid structure.

In conventional DNA nano-structures, split G-quartet sequences are highly stable, and it is difficult to maintain the split state; however, in the present invention, the pre-fixation on the opposite side (antiparallel type) successfully prevented the leakage of enzymatic activity. By using the technique of the present invention, the enzymatic activity can be controlled, for example, by varying the number of DNA strands attached to biomolecules. Unlike immunochromatography, the technique of the present invention is superior in that it does not require the fixation of antibodies or aptamers to underlying membranes or the like. In addition, the technique of the present invention, which can be composed only of nucleic acids, allows mass synthesis in a short time, cheaply and easily, with less lot differences compared to antibodies.

### Example 9: Preparation 7 of Nucleic Acid Structure

NanoLuc (Promega) is a luciferase with furimazine as a substrate, and its luminescence intensity is very strong, about 100 times the luminescence intensity of firefly luciferase. LgBiT (Promega) and SmBIT (Promega) are subunits of NanoLuc, and they are reconstituted as a luciferase by interacting with each other. LgBiT is a large subunit of approximately 18 kDa, and SmBiT is a small subunit of 11 amino acid residues. LgBiT and SmBiT have a dissociation constant of 190 μM and extremely low affinity, and they do not express luciferase activity when simply mixed (Andrew, D., (2016) ACS Chem. Biol. 11, 400-408). In this Example, only when the nucleic acid structure is structurally changed to the parallel type, SmBiT and LgBiT are brought close enough to interact, so that SmBiT and LgBiT interact only in the presence of a test substance, resulting in the exhibition of luciferase activity. Specifically, a nucleic acid structure was prepared in the following manner.

SmBiT and 95w (SEQ ID NO: 95) were bonded together. SmBIT (VTGYRLFEEIL-orn (N3)) modified with an azide group on the C-terminal side was chemically synthesized (Sigma-Aldrich). On the other hand, 95w modified with an amine on the 5-terminal side was chemically synthesized (IDT), and 1/30 equivalent (molar ratio) thereof was mixed with DBCO-NHS Ester (Click Chemistry Tools). Azide group-modified SmBiT and DBCO-modified 95w were mixed at a molar ratio of 1:1, and the mixture was allowed to stand for 16 hours and then purified by HPLC, thereby obtaining SmBiT-95w.

LgBiT and 138w (SEQ ID NO: 138) were bonded together. Lgbit was inserted into pH6HTN His6HaloTag T7 Vector (Promega) using the In-Fusion HD Cloning Kit (Clontech), and transformed into DH5α (SMOBIO Technology, Inc.) to obtain an LgBiT expression vector. The vector was transformed into BL21 (SMOBIO Technology, Inc.) and cultured in LB medium, and protein expression was induced with 0.4 mM of IPTG (Nacalai). The resultant was purified using the His-Spin Protein Miniprep Kit (Zymo Research), thereby obtaining HaloTag-fused LgBiT. On the other hand, 138w modified with an amine on the 5-terminal side was chemically synthesized (IDT), and 1/30 equivalent (molar ratio) thereof was mixed with HaloTag (registered trademark) Succinimidyl Ester (04) Ligand (Promega). The mixture was allowed to stand for 16 hours, and then purified by HPLC, thereby obtaining HaloTag ligand-binding 138w staple. HaloTag-fused LgBiT and HaloTag ligand-binding 138w staple were mixed at a molar ratio of 1:1, and the mixture was allowed to stand for 16 hours. Then, unreacted 138w staple was removed using a centrifugal ultrafiltration filter (Amicon Ultra 0.5 mL-50 K, Millipore), thereby obtaining LgBiT-138w.

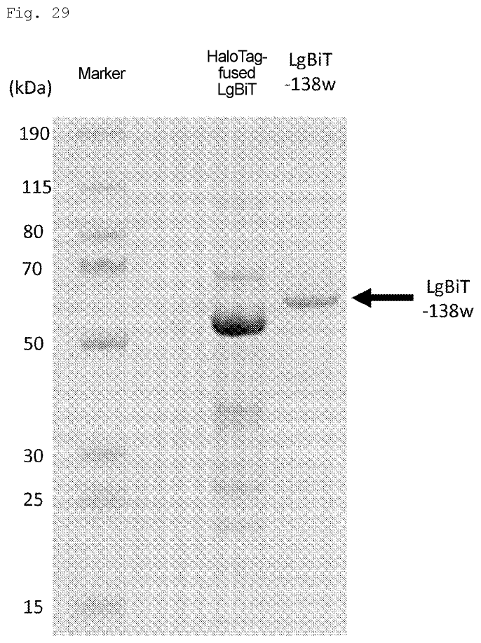

The obtained Lgbit-138w was confirmed by SDS polyacrylamide electrophoresis. FIG. 29 shows an electrophoretic image. From left, a marker (PageRuler Plus Prestain Protein Ladder, Thermo Scientific), HaloTag-fused LgBiT, and LgBiT-138w are shown. HaloTag-fused LgBiT migrated at a position similar to its expected molecular weight (approximately 54 kDa). In addition, the increase in molecular weight was confirmed based on the binding procedure of 138w shown above, suggesting that 138w bound to LgBiT.

A nucleic acid structure was prepared in the same manner as in Reference Example 1, except that some staple DNAs were changed to other staple DNAs. Tables 16 and 17 show the corresponding staple numbers of Reference Example 1, and the staple numbers, base sequences, and SEQ ID Nos of the staple DNAs used. LgBiT-138w was added at a molar ratio of 1:1 after the nucleic acid structure was prepared with other staple DNAs except for this. The configuration of the obtained nucleic acid structure was the same as that of the nucleic acid structure of Reference Example 1, except that the partial structure 1 and the partial structure 2 each contained a protruding structure consisting of single-stranded DNA of a telomere element sequence, the partial nucleic acid structure 1 contained a protruding structure containing a nucleic acid aptamer sequence (complementary sequence of miR-20 (Mature sequence hsa-miR-20a-5p: SEQ ID NO: 274)), and the partial nucleic acid structure 2 contained a sequence complementary to part of the nucleic acid aptamer sequence, and contained SmbiT and LgBiT.

TABLE 16

| Corresponding | | | |
|---|---|---|---|
| staple No. of | | | SEQ ID |
| Ref. Ex. 1 | staple No. | Sequence | No |
| 65 | 65long | TCTTAAACAGCTTGATGTGCCGTCGAGAGGGTGAGCCGCC | 345 |
| 66 | 66shortTelo | GTATAGCCGCTTTCGAGGTGAATTTTTTTAGGGTTAGGGT | 346 |
| 69 | 69long | ACAATGACAACAACCATAGCGGGGTTTTGCTCCAGGTCAG | 347 |
| 70 | 70shortTelo | GCGGATAAACCGATAGTTGCGCCGTTTTTAGGGTTAGGGT | 348 |
| 73 | 73LongCompM20 | GTGCAGGTAGTTTTGATATATTCGGTCGCTAGGCTGAGACTCC | 349 |
| | | TCACAAATAAA | |
| 74 | 74shortTelo | ATTAGGATTCGCCCACGCATAACCTTTTTAGGGTTAGGGT | 350 |
| 78s | 78s-telo | GTATTAAGGAGGCTTGCAGGGAGTTTTTTAGGGTTAGGGT | 351 |
| 166s | 166s-telo | GTCGAGGTAGAGATAGAACCCTTCTTTTTAGGGTTAGGGT | 352 |
| 169 | 169LongCompM20 | GTGCAGGTAGTTTTGGAACATTTCTGGCCAACGCCGTAAAGCA | 353 |
| | | CTAAAATCCTGTT | |
| 170 | 170shortTelo | CTAAAGGGCGACCAGTAATAAAAGTTTTTAGGGTTAGGGT | 354 |
| 173 | 173long | AGATTCACCAGTCACAAGCCCCCGATTTAGAGCGGTCCAC | 355 |
| 174 | 174shortTelo | GGAAAGCCGATTATTTACATTGGCTTTTTAGGGTTAGGGT | 356 |
| 177 | 177long | TCAATCGTCTGAAATGGGCGAACGTGGCGAGATCACCGCC | 357 |
| 178 | 178shortTelo | GAAGAAAGACCTACATTTTGACGCTTTTTAGGGTTAGGGT | 358 |

TABLE 17

| Corresponding | | | |
|---|---|---|---|
| staple No. of | | | SEQ ID |
| Ref. Ex. 1 | staple No. | Sequence | No |
| 81c | 81cLong | CAATATTTTTGAATGGTATAAACAGTTAATGCGTCATACA | 359 |
| 82s | 82sBothLongM20 | GTGCAGGTAGTTTTTAAAGGCCGCTTTTGCGAATACGTGGCAC | 360 |
| | | AGATTTTCTACCTGCACTATAAGCACTTTA | |
| 85c | 85cLong | CGCGAACTGATAGCCCAACGGGGTCAGTGCCTACTGGTAA | 361 |
| 86c | 86cShortMainM20 | AGTGCCCGCTATTAGTCTTTAATGTTTTCTACCTGCACTATAAG | 362 |
| | | CACTTTA | |
| 157c | 157cLong | AAACGGCTACAGAGGCTACGTGGACTCCAACGTAGTCCACT | 363 |
| 158c | 158cShortMainM20 | GAAAAACCTCGGAACGAGGGTAGCTTTTCTACCTGCACTATAA | 364 |
| | | GCACTTTA | |
| 161c | 161cLong | GCAGCGAAAGACAGCAGTCTATCAGGGCGATGTAGGGTTG | 365 |
| 162s | 162sBothLongM20 | GTGCAGGTAGTTTTTGACCTGAAAGCGTAAGGGATCGTCACCC | 366 |
| | | TCATTTTCTACCTGCACTATAAGCACTTTA | |
| 95w | SmBIT-95w | SmBIT-AAGGAATTGAGGAAGG | 367 |
| 138w | LgBiT-138w | LgBiT-ATCCGCGACCTGCTCC | 368 |

Staple DNA with a staple number labeled “Telo” contains a telomere element sequence (including two G triple repeats). The structure formed by the telomere element sequence, its position, etc. are the same as those in Reference Example 2. The telomere element sequence in the partial structure 1 and the telomere element sequence in the partial structure 2 can be linked in the presence of miR-20 and a monovalent cation to form a G-quartet structure.

Staple DNA with a staple number labeled “M20” contains the complementary sequence of miR-20 or a sequence complementary to part of the sequence. These sequences form a protruding structure consisting of single-stranded DNA without complementary binding to M13mp18. Among the three structures based on the conformation of the Holliday junction (FIG. 9: provided that Cross is an intermediate structure that mediates structural changes between Antiparallel and Parallel), when taking Antiparallel, the protruding structure in the partial structure 1 and the protruding structure in the partial structure 2 are arranged so that these protruding structures face each other (i.e., staple DNA containing the complementary sequence of miR-20 or a sequence complementary to part of the sequence is arranged). The complementary sequence of miR-20 in the partial structure 2 and the sequence complementary to part of the sequence in the partial structure 1 can be bonded based on complementary base pairing in the absence of miR-20.

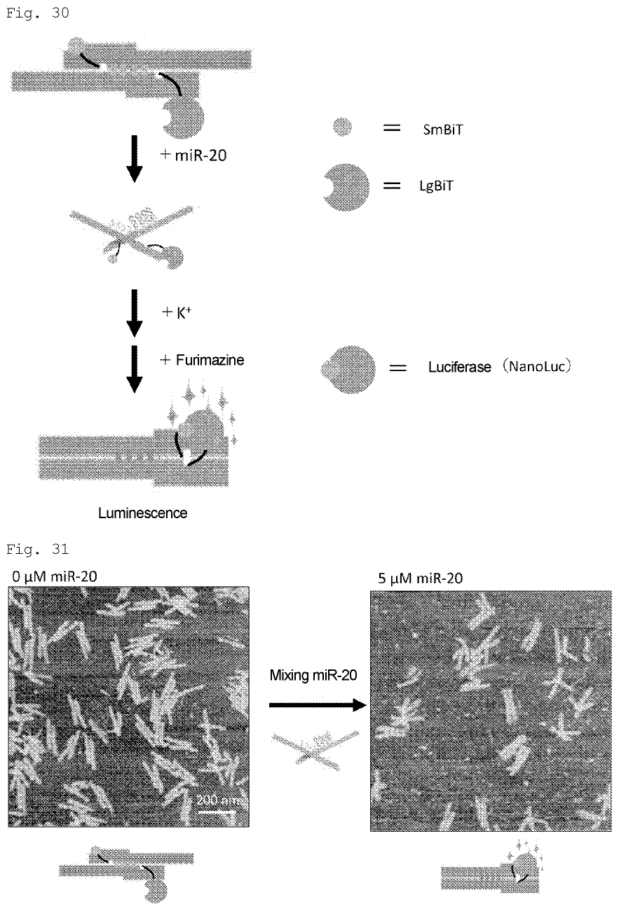

That is, as shown in FIG. 30, the nucleic acid structure of this Example is fixed at the antiparallel type based on complementary binding in the absence of miR-20, whereas in the presence of miR-20, the complementary bond dissociates to induce a structural change to the cross type, and the presence of a monovalent cation induces a structure change to the parallel type based on the formation of a G-quartet structure. As a result, SmBiT and LgBiT are in close proximity and interaction to be reconstituted as a luciferase.

A KCl solution (1 M KCl was dissolved in 1×TAE/Mg²⁺ test substance, miR20 (dissolved in RNase-free Water (catalog number: 9012, TakaRa) at 200 μM), was added, followed by still standing for 1 hour. AFM observation was then performed, and the presence of miR20 was visualized as a structural difference of the nucleic acid structure. The solution containing the nucleic acid structure contained 3.2 nM of the nucleic acid structure, 5 μM of miR20, and 200 mM of KCl. Further, each structure of the nucleic acid structure was counted from the acquired images, and its proportion was examined.

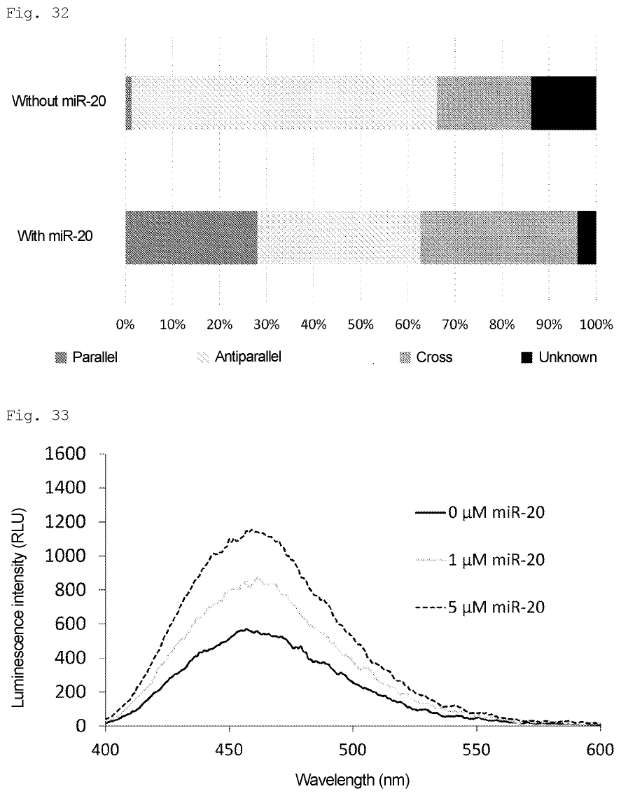

FIG. 31 shows AFM observation images. Further, FIG. 32 shows the proportion of each structure. The antiparallel type was the majority before the addition of miR-20, indicating that the addition of miR-20 induced a structural change from the antiparallel type to the parallel type.

### Example 10: Detection 6 of Enzymatic Activity

A KCl solution (1 M KCl was dissolved in 1×TAE/Mg²⁺ solution) was added to a solution containing the nucleic acid structure (Example 9), followed by still standing for 5 minutes. Then, a test substance, miR20 (dissolved in RNase-free Water (catalog number: 9012, TakaRa) at 200 μM), was added, followed by still standing for 15 minutes. The solution containing the nucleic acid structure contained 3.2 nM of the nucleic acid structure, 1 μM or 5 μM of miR20, and 200 mM of KCl. Then, furimazine (Nano-Glo Substrate) attached to the Nano-Glo HiBiT Blotting System (Promega) was added, and the luminescence intensity was quantified by a plate reader (SpectraMax iD5, Molecular Devices).

The results are shown in FIG. 33. The luminescence intensity increased when miR-20 was contained. This revealed that miR-20 can be detected using the nucleic acid structure.

SmBiT and LgBiT have a dissociation constant of about 190 μM and extremely low affinity, and they do not express luciferase activity when simply mixed (Andrew, D., (2016) ACS Chem. Biol. 11, 400-408). In the present invention, only when the nucleic acid structure was structurally changed to the parallel type, SmBiT and LgBiT were in close proximity and interaction to succeed in significantly increasing luciferase activity in the presence of a test substance. Further, the luciferase NanoLuc, which is reconstituted by the interaction of SmBiT and LgBiT, is known to have a luminescence intensity about 100 times that of firefly luciferase, and can be photographed with a smartphone camera (Arts, R. et al., (2016) Anal. Chem. 88, 4525-4532). Therefore, visualization is possible by increasing the concentration of the nucleic acid structure.

### SEQUENCE LISTING

1. A nucleic acid structure comprising a partial nucleic acid structure 1 and a partial nucleic acid structure 2,
the partial nucleic acid structure 1 and the partial nucleic acid structure 2 being linked by a Holliday junction,
the partial nucleic acid structure 1 comprising a protruding structure 1a containing a nucleic acid aptamer sequence, and a protruding structure 1b containing a split domain 1 of an enzyme, and
the partial nucleic acid structure 2 comprising a protruding structure 2a containing a sequence complementary to part or all of the nucleic acid aptamer sequence, and a protruding structure 2b containing a split domain 2 paired with the split domain 1.

2. The nucleic acid structure according to claim 1, wherein the partial structure 1 and the partial structure 2 each have an elongated shape.

3. The nucleic acid structure according to claim 1, which is made of a material containing a single-stranded circular nucleic acid and a staple nucleic acid containing a sequence complementary to the single-stranded circular nucleic acid.

4. The nucleic acid structure according to claim 1, wherein the protruding structures are arranged so that, among three structures (structural type 1, structural type 2, and structural type 3: provided that the structural type 2 is an intermediate structure that mediates structural changes between the structural type 1 and the structural type 3) based on the conformation of the Holliday junction, when taking the structural type 1, the protruding structure 1a and the protruding structure 2a face each other, and when taking the structural type 3, the protruding structure 1b and the protruding structure 2b face each other.

5. The nucleic acid structure according to claim 4, wherein the protruding structures are arranged so that the protruding structure 1a and the protruding structure 2a face each other and bind together based on complementary base pairing in the absence of a recognition substance for the nucleic acid aptamer sequence.

6. The nucleic acid structure according to claim 4, wherein the protruding structures are arranged so that the protruding structure 1b and the protruding structure 2b face each other to form the enzyme in the presence of a recognition substance for the nucleic acid aptamer sequence and in the presence of a monovalent cation.

7. The nucleic acid structure according to claim 1, wherein the enzyme is a nucleic acid enzyme containing two or more G-quartets.

8. The nucleic acid structure according to claim 1, wherein the recognition substance for the nucleic acid aptamer sequence is a biological material.

9. The nucleic acid structure according to claim 1, wherein the recognition substance for the nucleic acid aptamer sequence is at least one member selected from the group consisting of nucleic acids, proteins, peptides, low-molecular-weight compounds, physiologically active substances, and metal ions.

10. The nucleic acid structure according to claim 1, which is a nano-structure.

11. A test substance detection composition comprising the nucleic acid structure according to claim 1.

12. The test substance detection composition according to claim 11, which is a reagent or a test agent.

13. A method for detecting a test substance, comprising bringing the nucleic acid structure according to claim 1 into contact with a sample that may contain the test substance.


SEQUENCE LISTING (368 sequences)

SEQ ID NO: 1 - DNA, 38 bp, Artificial Sequence
  synthesized seqence, 1s
cctaatttttttatcctgaatcttaccaacgctatttt

SEQ ID NO: 2 - DNA, 28 bp, Artificial Sequence
  synthesized seqence, 2s
ttttacgagcgtctgcagcaccgttttt

SEQ ID NO: 3 - DNA, 32 bp, Artificial Sequence
  synthesized seqence, 3s
caagtttgaatgaaaccatcgatattccagag

SEQ ID NO: 4 - DNA, 32 bp, Artificial Sequence
  synthesized seqence, 4s
agctacaagccagttacaaaataaggccggaa

SEQ ID NO: 5 - DNA, 32 bp, Artificial Sequence
  synthesized seqence, 5s
acgtcacccctttagcgtcagactacaacgcc

SEQ ID NO: 6 - DNA, 32 bp, Artificial Sequence
  synthesized seqence, 6s
attatttaaagattagttgctattttgcaccc

SEQ ID NO: 7 - DNA, 32 bp, Artificial Sequence
  synthesized seqence, 7s
ttttcatcccattaccattagcaaacagccat

SEQ ID NO: 8 - DNA, 22 bp, Artificial Sequence
  synthesized seqence, 8s
ttgaagccttaaatctcccaat

SEQ ID NO: 9 - DNA, 28 bp, Artificial Sequence
  synthesized seqence, 9s
ccaaataagaaacgatttagagccagca

SEQ ID NO: 10 - DNA, 28 bp, Artificial Sequence
  synthesized seqence, 10
ttttaatcagtagcatagttagcgtttt

SEQ ID NO: 11 - DNA, 32 bp, Artificial Sequence
  synthesized seqence, 11
tcgtcttttccacagacagccctcgacagaat

SEQ ID NO: 12 - DNA, 22 bp, Artificial Sequence
  synthesized seqence, 12
tttttaacgatctaaagttttg

SEQ ID NO: 13 - DNA, 22 bp, Artificial Sequence
  synthesized seqence, 13s
aaatcaccagtagcaggcattt

SEQ ID NO: 14 - DNA, 32 bp, Artificial Sequence
  synthesized seqence, 14s
tgccatctgagccatttgggaatttttgttta

SEQ ID NO: 15 - DNA, 32 bp, Artificial Sequence
  synthesized seqence, 15s
gcgttttaaatgaaaatagcagcctcaccgtc

SEQ ID NO: 16 - DNA, 32 bp, Artificial Sequence
  synthesized seqence, 16s
accgactttttcataatcaaaatcctcatttt

SEQ ID NO: 17 - DNA, 42 bp, Artificial Sequence
  synthesized seqence, 17s
taaaaacaatagaaggcttatccggtattctaagaacgcgag

SEQ ID NO: 18 - DNA, 28 bp, Artificial Sequence
  synthesized seqence, 18s
aggtgaattatttacagagagaataaca

SEQ ID NO: 19 - DNA, 24 bp, Artificial Sequence
  synthesized seqence, 19s
accggaacggaaattattcattaa

SEQ ID NO: 20 - DNA, 32 bp, Artificial Sequence
  synthesized seqence, 20s
aatcagatgggaagcgcattagacgaaggtaa

SEQ ID NO: 21 - DNA, 32 bp, Artificial Sequence
  synthesized seqence, 21s
atattgaccgcctccctcagagccagaaccgc

SEQ ID NO: 22 - DNA, 32 bp, Artificial Sequence
  synthesized seqence, 22s
taactgaacattaccgcgcccaatagcaagca

SEQ ID NO: 23 - DNA, 32 bp, Artificial Sequence
  synthesized seqence, 23
tgtatggggtcaccagtacaaactgtagcgcg

SEQ ID NO: 24 - DNA, 32 bp, Artificial Sequence
  synthesized seqence, 24
tgtagcatccagacgttagtaaatgaattttc

SEQ ID NO: 25 - DNA, 32 bp, Artificial Sequence
  synthesized seqence, 25s
cagaaccgaaccgattgagggagggggagaat

SEQ ID NO: 26 - DNA, 32 bp, Artificial Sequence
  synthesized seqence, 26s
gtaggaatcaccctgaacaaagtccaaaaggg

SEQ ID NO: 27 - DNA, 32 bp, Artificial Sequence
  synthesized seqence, 27s
cgacattcccaccctcagagccacccgtactc

SEQ ID NO: 28 - DNA, 32 bp, Artificial Sequence
  synthesized seqence, 28s
attgagcgaagcaagccgtttttattttcatc

SEQ ID NO: 29 - DNA, 32 bp, Artificial Sequence
  synthesized seqence, 29s
gagccgccttaccagcgccaaagaagagggta

SEQ ID NO: 30 - DNA, 32 bp, Artificial Sequence
  synthesized seqence, 30s
tcgagaacctaatatcagagagatagaaaatt

SEQ ID NO: 31 - DNA, 32 bp, Artificial Sequence
  synthesized seqence, 31s
catatggtaccagaaccaccaccatgatataa

SEQ ID NO: 32 - DNA, 32 bp, Artificial Sequence
  synthesized seqence, 32s
agaattgagtattaaaccaagtaccgcactca

SEQ ID NO: 33 - DNA, 30 bp, Artificial Sequence
  synthesized seqence, 33
tcggtcatgtaacactgagtttcattttgc

SEQ ID NO: 34 - DNA, 28 bp, Artificial Sequence
  synthesized seqence, 34
cccatgtaccagcccccttattagcgtt

SEQ ID NO: 35 - DNA, 26 bp, Artificial Sequence
  synthesized seqence, 35
taaacaactttcaacagtttcagcgg

SEQ ID NO: 36 - DNA, 32 bp, Artificial Sequence
  synthesized seqence, 36s
gccagcatttttgtcacaatcaataacccaca

SEQ ID NO: 37 - DNA, 32 bp, Artificial Sequence
  synthesized seqence, 37s
aagaacgggttaagcccaataataccacggaa

SEQ ID NO: 38 - DNA, 32 bp, Artificial Sequence
  synthesized seqence, 38s
taagtttatgacaggaggttgaggagtaccag

SEQ ID NO: 39 - DNA, 32 bp, Artificial Sequence
  synthesized seqence, 39s
aaacaatgtcggctgtctttccttatcattcc

SEQ ID NO: 40 - DNA, 32 bp, Artificial Sequence
  synthesized seqence, 40s
acgattggaagaaacgcaaagacaagagcaag

SEQ ID NO: 41 - DNA, 24 bp, Artificial Sequence
  synthesized seqence, 41
agtgagaagcaagcccaataggaa

SEQ ID NO: 42 - DNA, 32 bp, Artificial Sequence
  synthesized seqence, 42s
atcaataaaaatagcaatagctataggtggca

SEQ ID NO: 43 - DNA, 32 bp, Artificial Sequence
  synthesized seqence, 43s
acatataaccttgatattcacaaaagagaagg

SEQ ID NO: 44 - DNA, 32 bp, Artificial Sequence
  synthesized seqence, 44s
agcccttttaatttacgagcatgtagaaacca

SEQ ID NO: 45 - DNA, 32 bp, Artificial Sequence
  synthesized seqence, 45s
tcctcattgaaaatacatacataacttaccga

SEQ ID NO: 46 - DNA, 40 bp, Artificial Sequence
  synthesized seqence, 46s
aaataatatcccatccttaagaaaagtaagcatatgttag

SEQ ID NO: 47 - DNA, 32 bp, Artificial Sequence
  synthesized seqence, 47s
caaacgtaaaagccagaatggaaaacatgaaa

SEQ ID NO: 48 - DNA, 26 bp, Artificial Sequence
  synthesized seqence, 48s
gaatcataataaggcgttaaataaga

SEQ ID NO: 49 - DNA, 32 bp, Artificial Sequence
  synthesized seqence, 49s
aacaaagtaagtcctgaacaagaaaaacaccg

SEQ ID NO: 50 - DNA, 32 bp, Artificial Sequence
  synthesized seqence, 50s
tctgaattactccttattacgcaggatagccg

SEQ ID NO: 51 - DNA, 32 bp, Artificial Sequence
  synthesized seqence, 51s
tgtgataaattactagaaaaagcctttatcaa

SEQ ID NO: 52 - DNA, 28 bp, Artificial Sequence
  synthesized seqence, 52
agccaccaccaccggaaccagagccacc

SEQ ID NO: 53 - DNA, 34 bp, Artificial Sequence
  synthesized seqence, 53
cagggatatagaaaggaacaactaaaggaattgc

SEQ ID NO: 54 - DNA, 32 bp, Artificial Sequence
  synthesized seqence, 54
gaataataattttttcgaaccgccaccctcag

SEQ ID NO: 55 - DNA, 32 bp, Artificial Sequence
  synthesized seqence, 55s
caatagattaccagaaggaaaccgaactggca

SEQ ID NO: 56 - DNA, 32 bp, Artificial Sequence
  synthesized seqence, 56s
tgattaagtaccgttccagtaagccccctgcc

SEQ ID NO: 57 - DNA, 32 bp, Artificial Sequence
  synthesized seqence, 57
gctccaaatagtaccgccaccctcgccaccct

SEQ ID NO: 58 - DNA, 32 bp, Artificial Sequence
  synthesized seqence, 58
caccctcaacgttgaaaatctccaaaaaaaag

SEQ ID NO: 59 - DNA, 32 bp, Artificial Sequence
  synthesized seqence, 59s
atcatatgtttaatggtttgaaataccgaccg

SEQ ID NO: 60 - DNA, 32 bp, Artificial Sequence
  synthesized seqence, 60s
caataataatgcagaacgcgcctgtgtttagt

SEQ ID NO: 61 - DNA, 32 bp, Artificial Sequence
  synthesized seqence, 61
atcagcttcggaataggtgtatcacaccctca

SEQ ID NO: 62 - DNA, 32 bp, Artificial Sequence
  synthesized seqence, 62
aggaggttaggagcctttaattgtatcggttt

SEQ ID NO: 63 - DNA, 32 bp, Artificial Sequence
  synthesized seqence, 63s
tggcttttacggaatacccaaaagaggaaacg

SEQ ID NO: 64 - DNA, 32 bp, Artificial Sequence
  synthesized seqence, 64s
gacctaaacgttatacaaattcttacaacatg

SEQ ID NO: 65 - DNA, 32 bp, Artificial Sequence
  synthesized seqence, 65
agcttgatgtgccgtcgagagggtgagccgcc

SEQ ID NO: 66 - DNA, 32 bp, Artificial Sequence
  synthesized seqence, 66
gtatagccgctttcgaggtgaatttcttaaac

SEQ ID NO: 67 - DNA, 32 bp, Artificial Sequence
  synthesized seqence, 67s
ttcagctaaggtttaacgtcagataattgcgt

SEQ ID NO: 68 - DNA, 32 bp, Artificial Sequence
  synthesized seqence, 68s
aaagccaaatattttagttaatttcatcttct

SEQ ID NO: 69 - DNA, 32 bp, Artificial Sequence
  synthesized seqence, 69
aacaaccatagcggggttttgctccaggtcag

SEQ ID NO: 70 - DNA, 32 bp, Artificial Sequence
  synthesized seqence, 70
gcggataaaccgatagttgcgccgacaatgac

SEQ ID NO: 71 - DNA, 32 bp, Artificial Sequence
  synthesized seqence, 71s
agtaacagcagacgacgacaataaaccagtat

SEQ ID NO: 72 - DNA, 32 bp, Artificial Sequence
  synthesized seqence, 72s
tttcaaatcgctcaacagtagggcgtaaagta

SEQ ID NO: 73 - DNA, 32 bp, Artificial Sequence
  synthesized seqence, 73
cggtcgctaggctgagactcctcacaaataaa

SEQ ID NO: 74 - DNA, 32 bp, Artificial Sequence
  synthesized seqence, 74
attaggattcgcccacgcataaccgatatatt

SEQ ID NO: 75 - DNA, 32 bp, Artificial Sequence
  synthesized seqence, 75s
attctgtctaccttttacatcgggaaaattat

SEQ ID NO: 76 - DNA, 32 bp, Artificial Sequence
  synthesized seqence, 76s
gaatcgccaagacaaagaacgcgagaaaactt

SEQ ID NO: 77 - DNA, 32 bp, Artificial Sequence
  synthesized seqence, 77s
tatttcggaacctattattctgaagcgcagtc

SEQ ID NO: 78 - DNA, 24 bp, Artificial Sequence
  synthesized seqence, 78s
gtattaaggaggcttgcagggagt

SEQ ID NO: 79 - DNA, 32 bp, Artificial Sequence
  synthesized seqence, 79s
taacggataaagtaccgacaaaagttaattga

SEQ ID NO: 80 - DNA, 32 bp, Artificial Sequence
  synthesized seqence, 80s
ccaatcgcatatttaacaacgccaagtaataa

SEQ ID NO: 81 - DNA, 32 bp, Artificial Sequence
  synthesized seqence, 81c
ttgaatggtataaacagttaatgcgtcataca

SEQ ID NO: 82 - DNA, 40 bp, Artificial Sequence
  synthesized seqence, 82s
taaaggccgcttttgcgaatacgtggcacagacaatattt

SEQ ID NO: 83 - DNA, 32 bp, Artificial Sequence
  synthesized seqence, 83w
agattttcgatgatacaggagtgttgagtaac

SEQ ID NO: 84 - DNA, 32 bp, Artificial Sequence
  synthesized seqence, 84w
taagttttaaaacagaaataaagagaatatac

SEQ ID NO: 85 - DNA, 32 bp, Artificial Sequence
  synthesized seqence, 85c
gatagcccaacggggtcagtgcctactggtaa

SEQ ID NO: 86 - DNA, 32 bp, Artificial Sequence
  synthesized seqence, 86c
agtgcccgctattagtctttaatgcgcgaact

SEQ ID NO: 87 - DNA, 32 bp, Artificial Sequence
  synthesized seqence, 87w
ttgcacgtggtgaggcggtcagtacagaagat

SEQ ID NO: 88 - DNA, 32 bp, Artificial Sequence
  synthesized seqence, 88w
gcctgcaatagaacctaccatatcagaaacaa

SEQ ID NO: 89 - DNA, 19 bp, Artificial Sequence
  synthesized seqence, 89w
cgaaccaccagttaacacc

SEQ ID NO: 90 - DNA, 19 bp, Artificial Sequence
  synthesized seqence, 90w
aaaacagataaaacatcgc

SEQ ID NO: 91 - DNA, 16 bp, Artificial Sequence
  synthesized seqence, 91w
cattaaaaataccgaa

SEQ ID NO: 92 - DNA, 24 bp, Artificial Sequence
  synthesized seqence, 92w
ggaagggtcagtgccacgctgaga

SEQ ID NO: 93 - DNA, 32 bp, Artificial Sequence
  synthesized seqence, 93w
caaatgaagtttggattatacttcaataccaa

SEQ ID NO: 94 - DNA, 24 bp, Artificial Sequence
  synthesized seqence, 94w
gcaaatcaacagttgagccagcag

SEQ ID NO: 95 - DNA, 16 bp, Artificial Sequence
  synthesized seqence, 95w
aaggaattgaggaagg

SEQ ID NO: 96 - DNA, 32 bp, Artificial Sequence
  synthesized seqence, 96w
tcctgattaaatctaaagcatcacaatatctg

SEQ ID NO: 97 - DNA, 32 bp, Artificial Sequence
  synthesized seqence, 97w
acctcaaagatgatggcaattcatttattcat

SEQ ID NO: 98 - DNA, 32 bp, Artificial Sequence
  synthesized seqence, 98w
ctaacaactatcaaaccctcaatccttgctga

SEQ ID NO: 99 - DNA, 32 bp, Artificial Sequence
  synthesized seqence, 99w
gtcagttgttatctaaaatatctttaggagca

SEQ ID NO: 100 - DNA, 32 bp, Artificial Sequence
  synthesized seqence, 100w
gattatcaacgttattaattttaataaatcct

SEQ ID NO: 101 - DNA, 32 bp, Artificial Sequence
  synthesized seqence, 101w
gtaacattgagcggaattatcatctgatgaaa

SEQ ID NO: 102 - DNA, 32 bp, Artificial Sequence
  synthesized seqence, 102w
taatacatattcgacaactcgtataagtttga

SEQ ID NO: 103 - DNA, 32 bp, Artificial Sequence
  synthesized seqence, 103w
ttgcccgataatagattagagccgtcaataga

SEQ ID NO: 104 - DNA, 36 bp, Artificial Sequence
  synthesized seqence, 104w
accagaagatcattttgcggaacattttttagactt

SEQ ID NO: 105 - DNA, 36 bp, Artificial Sequence
  synthesized seqence, 105s
aacaatttaagaaaacaaaattaattttaagaaacc

SEQ ID NO: 106 - DNA, 28 bp, Artificial Sequence
  synthesized seqence, 106w
tacaaacattgaggatttagaagttttt

SEQ ID NO: 107 - DNA, 32 bp, Artificial Sequence
  synthesized seqence, 107s
caaacatccatttgaattacctttaacatagc

SEQ ID NO: 108 - DNA, 32 bp, Artificial Sequence
  synthesized seqence, 108w
aaacagtacctgagcaaaagaagaatattcct

SEQ ID NO: 109 - DNA, 32 bp, Artificial Sequence
  synthesized seqence, 109s
ttcaattacataaatcaatatatgctattaat

SEQ ID NO: 110 - DNA, 32 bp, Artificial Sequence
  synthesized seqence, 110w
taaccttgatcgcgcagaggcgaacaatataa

SEQ ID NO: 111 - DNA, 32 bp, Artificial Sequence
  synthesized seqence, 111s
gagaatattcgcctgattgctttgtgaataat

SEQ ID NO: 112 - DNA, 32 bp, Artificial Sequence
  synthesized seqence, 112s
tttaggcactatatgtaaatgctgatgcaaat

SEQ ID NO: 113 - DNA, 32 bp, Artificial Sequence
  synthesized seqence, 113s
gttacaaagaggcattttcgagccacatgtaa

SEQ ID NO: 114 - DNA, 32 bp, Artificial Sequence
  synthesized seqence, 114s
ttatataacttctgtaaatcgtcgtgagtgaa

SEQ ID NO: 115 - DNA, 32 bp, Artificial Sequence
  synthesized seqence, 115s
taattttcttttaacctccggcttaggttggg

SEQ ID NO: 116 - DNA, 32 bp, Artificial Sequence
  synthesized seqence, 116s
gactacctccttagaatccttgaatttaatgg

SEQ ID NO: 117 - DNA, 32 bp, Artificial Sequence
  synthesized seqence, 117s
gatagctttttatcaaaatcataggtctgaga

SEQ ID NO: 118 - DNA, 42 bp, Artificial Sequence
  synthesized seqence, 118s
gagtcaatagtgaaagattaagacgctgagttttttacattt

SEQ ID NO: 119 - DNA, 42 bp, Artificial Sequence
  synthesized seqence, 119s
accataaatcaaaagttcagaaaacgagaattttaaaatgtt

SEQ ID NO: 120 - DNA, 32 bp, Artificial Sequence
  synthesized seqence, 120s
tttaaacaatcaggtctttaccctgactatta

SEQ ID NO: 121 - DNA, 32 bp, Artificial Sequence
  synthesized seqence, 121s
tagtcagaattcattgaatccccccggaatcg

SEQ ID NO: 122 - DNA, 32 bp, Artificial Sequence
  synthesized seqence, 122s
gagtagatagcaaagcggattgcatcaaaaag

SEQ ID NO: 123 - DNA, 28 bp, Artificial Sequence
  synthesized seqence, 123w
agaccaggaaagaggacagatgaatttt

SEQ ID NO: 124 - DNA, 36 bp, Artificial Sequence
  synthesized seqence, 124w
agtgaatatcaacgtaacaaagctttttcggtgtac

SEQ ID NO: 125 - DNA, 42 bp, Artificial Sequence
  synthesized seqence, 125s
aaaagaagttttgcatagcgagaggcttttttttgctcattc

SEQ ID NO: 126 - DNA, 32 bp, Artificial Sequence
  synthesized seqence, 126w
caactttgcgcataggctggctgaatattcat

SEQ ID NO: 127 - DNA, 32 bp, Artificial Sequence
  synthesized seqence, 127w
tacccaaaaggcttgccctgacgagacgataa

SEQ ID NO: 128 - DNA, 24 bp, Artificial Sequence
  synthesized seqence, 128s
aaaccaaacagagggggtaatagt

SEQ ID NO: 129 - DNA, 32 bp, Artificial Sequence
  synthesized seqence, 129w
aagagtaacaatcataagggaaccgaactgac

SEQ ID NO: 130 - DNA, 32 bp, Artificial Sequence
  synthesized seqence, 130w
agaacgagtcttgacaagaaccggccttcatc

SEQ ID NO: 131 - DNA, 32 bp, Artificial Sequence
  synthesized seqence, 131w
tagactggccctcgtttaccagacgaaacacc

SEQ ID NO: 132 - DNA, 32 bp, Artificial Sequence
  synthesized seqence, 132w
cagacggtacaaagtacaacggagttatacca

SEQ ID NO: 133 - DNA, 32 bp, Artificial Sequence
  synthesized seqence, 133w
agcgcgaatagtaaattgggcttgagcaacac

SEQ ID NO: 134 - DNA, 32 bp, Artificial Sequence
  synthesized seqence, 134s
tatcataaatagcgtccaatactgtcaaatgc

SEQ ID NO: 135 - DNA, 32 bp, Artificial Sequence
  synthesized seqence, 135w
catcgcctatgttacttagccggaacgaggcg

SEQ ID NO: 136 - DNA, 32 bp, Artificial Sequence
  synthesized seqence, 136w
taatttcactttgacccccagcgaatttgtat

SEQ ID NO: 137 - DNA, 32 bp, Artificial Sequence
  synthesized seqence, 137w
tcataaattacgaggcatagtaagagatggtt

SEQ ID NO: 138 - DNA, 16 bp, Artificial Sequence
  synthesized seqence, 138w
atccgcgacctgctcc

SEQ ID NO: 139 - DNA, 24 bp, Artificial Sequence
  synthesized seqence, 139w
gataaattgtgtcgaaacactaaa

SEQ ID NO: 140 - DNA, 32 bp, Artificial Sequence
  synthesized seqence, 140w
acactcatactttaatcattgtgataacgcca

SEQ ID NO: 141 - DNA, 32 bp, Artificial Sequence
  synthesized seqence, 141s
aaaggaatttagtttgaccattagctgcgaac

SEQ ID NO: 142 - DNA, 24 bp, Artificial Sequence
  synthesized seqence, 142w
atgcgattaaagaggcaaaagaat

SEQ ID NO: 143 - DNA, 32 bp, Artificial Sequence
  synthesized seqence, 143s
cgcaaatgaactaatgcagatacaattacctt

SEQ ID NO: 144 - DNA, 16 bp, Artificial Sequence
  synthesized seqence, 144w
agactttttcatgagg

SEQ ID NO: 145 - DNA, 19 bp, Artificial Sequence
  synthesized seqence, 145w
aagtttccattgcaccaac

SEQ ID NO: 146 - DNA, 32 bp, Artificial Sequence
  synthesized seqence, 146w
ctaaaacgttaagaactggctcattaggaata

SEQ ID NO: 147 - DNA, 32 bp, Artificial Sequence
  synthesized seqence, 147s
ccacattcgtcaataacctgtttagtttcatt

SEQ ID NO: 148 - DNA, 19 bp, Artificial Sequence
  synthesized seqence, 148w
aaaatacgttgaggactaa

SEQ ID NO: 149 - DNA, 32 bp, Artificial Sequence
  synthesized seqence, 149w
tcaggacgtaatgccactacgaagaaacgggt

SEQ ID NO: 150 - DNA, 32 bp, Artificial Sequence
  synthesized seqence, 150s
ttcatttgttcatcagttgagatttataccag

SEQ ID NO: 151 - DNA, 32 bp, Artificial Sequence
  synthesized seqence, 151w
attaaagattgggaagaaaaatctattacagg

SEQ ID NO: 152 - DNA, 32 bp, Artificial Sequence
  synthesized seqence, 152s
tagaaagagggcgcgagctgaaaattaaatat

SEQ ID NO: 153 - DNA, 32 bp, Artificial Sequence
  synthesized seqence, 153w
aaaacgaatccagtttggaacaagcaaagggc

SEQ ID NO: 154 - DNA, 32 bp, Artificial Sequence
  synthesized seqence, 154s
caattctactaacggaacaacattacgttaat

SEQ ID NO: 155 - DNA, 32 bp, Artificial Sequence
  synthesized seqence, 155s
agtgttgttgaggggacgacgacacgtgcatc

SEQ ID NO: 156 - DNA, 32 bp, Artificial Sequence
  synthesized seqence, 156s
tgccagttctaatagtagtagcatttgctgaa

SEQ ID NO: 157 - DNA, 32 bp, Artificial Sequence
  synthesized seqence, 157c
cagaggctacgtggactccaacgtagtccact

SEQ ID NO: 158 - DNA, 32 bp, Artificial Sequence
  synthesized seqence, 158c
gaaaaacctcggaacgagggtagcaacggcta

SEQ ID NO: 159 - DNA, 32 bp, Artificial Sequence
  synthesized seqence, 159s
aataaatcatgggcgcatcgtaacgtatcggc

SEQ ID NO: 160 - DNA, 32 bp, Artificial Sequence
  synthesized seqence, 160s
ctcaggaaaaagaatagcccgagagcccacta

SEQ ID NO: 161 - DNA, 32 bp, Artificial Sequence
  synthesized seqence, 161c
agacagcagtctatcagggcgatgtagggttg

SEQ ID NO: 162 - DNA, 40 bp, Artificial Sequence
  synthesized seqence, 162s
tgacctgaaagcgtaagggatcgtcaccctcagcagcgaa

SEQ ID NO: 163 - DNA, 40 bp, Artificial Sequence
  synthesized seqence, 163s
gaattagcaaaattaatgaccgtaatgggatagctttccg

SEQ ID NO: 164 - DNA, 32 bp, Artificial Sequence
  synthesized seqence, 164s
ataaatcagatcgcactccagccaggtcacgt

SEQ ID NO: 165 - DNA, 32 bp, Artificial Sequence
  synthesized seqence, 165s
cgtgaaccatcacccaaatcaagtaatccctt

SEQ ID NO: 166 - DNA, 24 bp, Artificial Sequence
  synthesized seqence, 166s
gtcgaggtagagatagaacccttc

SEQ ID NO: 167 - DNA, 32 bp, Artificial Sequence
  synthesized seqence, 167s
gcaccgctgttccgaaatcggcaatttttggg

SEQ ID NO: 168 - DNA, 32 bp, Artificial Sequence
  synthesized seqence, 168s
ctaaatcgcgtcggattctccgtgaggcaaag

SEQ ID NO: 169 - DNA, 32 bp, Artificial Sequence
  synthesized seqence, 169
tggccaacgccgtaaagcactaaaatcctgtt

SEQ ID NO: 170 - DNA, 32 bp, Artificial Sequence
  synthesized seqence, 170
ctaaagggcgaccagtaataaaagggacattc

SEQ ID NO: 171 - DNA, 32 bp, Artificial Sequence
  synthesized seqence, 171s
cgccattcgccccagcaggcgaaatcggaacc

SEQ ID NO: 172 - DNA, 32 bp, Artificial Sequence
  synthesized seqence, 172s
tgatggtgtctggtgccggaaaccggaacaaa

SEQ ID NO: 173 - DNA, 32 bp, Artificial Sequence
  synthesized seqence, 173
cagtcacaagcccccgatttagagcggtccac

SEQ ID NO: 174 - DNA, 32 bp, Artificial Sequence
  synthesized seqence, 174
ggaaagccgattatttacattggcagattcac

SEQ ID NO: 175 - DNA, 32 bp, Artificial Sequence
  synthesized seqence, 175s
ggaagggcagagagttgcagcaagcttgacgg

SEQ ID NO: 176 - DNA, 32 bp, Artificial Sequence
  synthesized seqence, 176s
gctggtttgccattcaggctgcgctgagcgag

SEQ ID NO: 177 - DNA, 32 bp, Artificial Sequence
  synthesized seqence, 177
ctgaaatgggcgaacgtggcgagatcaccgcc

SEQ ID NO: 178 - DNA, 32 bp, Artificial Sequence
  synthesized seqence, 178
gaagaaagacctacattttgacgctcaatcgt

SEQ ID NO: 179 - DNA, 32 bp, Artificial Sequence
  synthesized seqence, 179s
tacgccagaacagctgattgccctaaggaagg

SEQ ID NO: 180 - DNA, 32 bp, Artificial Sequence
  synthesized seqence, 180s
tggccctggatcggtgcgggcctcttcctgta

SEQ ID NO: 181 - DNA, 32 bp, Artificial Sequence
  synthesized seqence, 181
atggaaatcgaaaggagcgggcgccaccagtg

SEQ ID NO: 182 - DNA, 32 bp, Artificial Sequence
  synthesized seqence, 182
tggcaagtgccattgcaacaggaaaaacgctc

SEQ ID NO: 183 - DNA, 32 bp, Artificial Sequence
  synthesized seqence, 183s
aggcgattgggtggtttttctttttagggcgc

SEQ ID NO: 184 - DNA, 32 bp, Artificial Sequence
  synthesized seqence, 184s
agacgggcctggcgaaagggggatggaacgcc

SEQ ID NO: 185 - DNA, 32 bp, Artificial Sequence
  synthesized seqence, 185
taccgccagtagcggtcacgctgctgcgtatt

SEQ ID NO: 186 - DNA, 32 bp, Artificial Sequence
  synthesized seqence, 186
accacacctgctggtaatatccagaacaatat

SEQ ID NO: 187 - DNA, 32 bp, Artificial Sequence
  synthesized seqence, 187s
ccagtcacgcggggagaggcggttgcgtaacc

SEQ ID NO: 188 - DNA, 32 bp, Artificial Sequence
  synthesized seqence, 188s
gggcgccaaagttgggtaacgccattttgtta

SEQ ID NO: 189 - DNA, 32 bp, Artificial Sequence
  synthesized seqence, 189
ctcaaactatcggcctcgccgcgcttaatgcg

SEQ ID NO: 190 - DNA, 34 bp, Artificial Sequence
  synthesized seqence, 190
tatggttgtaacatcacttgcctgagtagaagaa

SEQ ID NO: 191 - DNA, 28 bp, Artificial Sequence
  synthesized seqence, 191
ccgctacaggagctgcattaatgaatcg

SEQ ID NO: 192 - DNA, 32 bp, Artificial Sequence
  synthesized seqence, 192s
atgcctgcggaaacctgtcgtgccgcgcgtac

SEQ ID NO: 193 - DNA, 24 bp, Artificial Sequence
  synthesized seqence, 193s
gccaacgcgacgttgtaaaacgac

SEQ ID NO: 194 - DNA, 28 bp, Artificial Sequence
  synthesized seqence, 194s
ggccagtgcctaaattgtaaacgttaat

SEQ ID NO: 195 - DNA, 42 bp, Artificial Sequence
  synthesized seqence, 195s
attttgttttcaaaagggtgagaaaggccggagacagtcaaa

SEQ ID NO: 196 - DNA, 32 bp, Artificial Sequence
  synthesized seqence, 196s
ggtaaagaaaaattcgcattaaatgggttttc

SEQ ID NO: 197 - DNA, 32 bp, Artificial Sequence
  synthesized seqence, 197s
aatcagctaatgcaatgcctgagtaatgtgta

SEQ ID NO: 198 - DNA, 32 bp, Artificial Sequence
  synthesized seqence, 198s
atattttacattttttaaccaatagtgctgca

SEQ ID NO: 199 - DNA, 32 bp, Artificial Sequence
  synthesized seqence, 199s
atcaaaaagataaaaatttttagaaccctcat

SEQ ID NO: 200 - DNA, 32 bp, Artificial Sequence
  synthesized seqence, 200s
aacgcaagtaattcgcgtctggccttcgctat

SEQ ID NO: 201 - DNA, 32 bp, Artificial Sequence
  synthesized seqence, 201s
gccagcttttttgcgggagaagcctttatttc

SEQ ID NO: 202 - DNA, 32 bp, Artificial Sequence
  synthesized seqence, 202s
tgtaatactcatcaacattaaatgaactgttg

SEQ ID NO: 203 - DNA, 32 bp, Artificial Sequence
  synthesized seqence, 203s
taacaaccgttgtaccaaaaacattatgaccc

SEQ ID NO: 204 - DNA, 24 bp, Artificial Sequence
  synthesized seqence, 204
attagtaactttgacgagcacgta

SEQ ID NO: 205 - DNA, 26 bp, Artificial Sequence
  synthesized seqence, 205
ttaaccgttgtagcaatacttctttg

SEQ ID NO: 206 - DNA, 30 bp, Artificial Sequence
  synthesized seqence, 206
aattgcgttcctcgttagaatcaacgcaaa

SEQ ID NO: 207 - DNA, 28 bp, Artificial Sequence
  synthesized seqence, 207
taacgtgctttgcgctcactgcccgctt

SEQ ID NO: 208 - DNA, 32 bp, Artificial Sequence
  synthesized seqence, 208s
tccagtcgaggtcgactctagaggccaaaaac

SEQ ID NO: 209 - DNA, 32 bp, Artificial Sequence
  synthesized seqence, 209s
tcaccatctgtataagcaaatattaagcttgc

SEQ ID NO: 210 - DNA, 22 bp, Artificial Sequence
  synthesized seqence, 210s
accgagctcgaattctcacatt

SEQ ID NO: 211 - DNA, 28 bp, Artificial Sequence
  synthesized seqence, 211s
ttgataatcagaaaagccatccccgggt

SEQ ID NO: 212 - DNA, 34 bp, Artificial Sequence
  synthesized seqence, 212s
aggaagataatatgatattcaaccgttctagctg

SEQ ID NO: 213 - DNA, 32 bp, Artificial Sequence
  synthesized seqence, 213s
gagctaacgtaatcatggtcatagcatgtcaa

SEQ ID NO: 214 - DNA, 22 bp, Artificial Sequence
  synthesized seqence, 214s
ataaattaatgccggaccccgg

SEQ ID NO: 215 - DNA, 32 bp, Artificial Sequence
  synthesized seqence, 215s
tcatatgtagagggtagctatttttgagagat

SEQ ID NO: 216 - DNA, 32 bp, Artificial Sequence
  synthesized seqence, 216s
tgtgtgaataaagcctggggtgccggaggccg

SEQ ID NO: 217 - DNA, 32 bp, Artificial Sequence
  synthesized seqence, 217s
ctacaaaggtaatcgtaaaactagctgtttcc

SEQ ID NO: 218 - DNA, 32 bp, Artificial Sequence
  synthesized seqence, 218s
ataaagtgattgttatccgctcacagagaatc

SEQ ID NO: 219 - DNA, 38 bp, Artificial Sequence
  synthesized seqence, 219s
gatgaacggctatcaggtcattgcctgagagtcttttt

SEQ ID NO: 220 - DNA, 28 bp, Artificial Sequence
  synthesized seqence, 220s
ttttggagcaaacaaattccacactttt

SEQ ID NO: 221 - DNA, 32 bp, Artificial Sequence
  synthesized seqence, 221
tgtccatcgagcgggagctaaacataatgagt

SEQ ID NO: 222 - DNA, 32 bp, Artificial Sequence
  synthesized seqence, 222
attaaagggtgaggccaccgagtaaaagagtc

SEQ ID NO: 223 - DNA, 32 bp, Artificial Sequence
  synthesized seqence, 223s
cggcggatgcaataaagcctcagagcataaag

SEQ ID NO: 224 - DNA, 32 bp, Artificial Sequence
  synthesized seqence, 224s
tggtgtagatacaggcaaggcaaagaggtcat

SEQ ID NO: 225 - DNA, 24 bp, Artificial Sequence
  synthesized seqence, 225s
ttttgcggttaattgctccttttg

SEQ ID NO: 226 - DNA, 32 bp, Artificial Sequence
  synthesized seqence, 226s
gagtacctatggcttagagcttaataacatcc

SEQ ID NO: 227 - DNA, 32 bp, Artificial Sequence
  synthesized seqence, 227s
tataatgcaaactccaacaggtcaggattaga

SEQ ID NO: 228 - DNA, 32 bp, Artificial Sequence
  synthesized seqence, 228s
ccggaagctgtagctcaacatgttggtggcat

SEQ ID NO: 229 - DNA, 32 bp, Artificial Sequence
  synthesized seqence, 229s
gcaactaaattcgagcttcaaagcgaaccaga

SEQ ID NO: 230 - DNA, 32 bp, Artificial Sequence
  synthesized seqence, 230s
gcgttttaagtacggtgtctggaagctatatt

SEQ ID NO: 231 - DNA, 32 bp, Artificial Sequence
  synthesized seqence, 231s
ccatataagaagcccgaaagacttcaaatatc

SEQ ID NO: 232 - DNA, 32 bp, Artificial Sequence
  synthesized seqence, 232s
attaagagcagttgattcccaattatacattt

SEQ ID NO: 233 - DNA, 32 bp, Artificial Sequence
  synthesized seqence, 233
tataatcagattttagacaggaacccggaagc

SEQ ID NO: 234 - DNA, 22 bp, Artificial Sequence
  synthesized seqence, 234
ttttaatcctgagaagtgtttt

SEQ ID NO: 235 - DNA, 28 bp, Artificial Sequence
  synthesized seqence, 235
ttttaacatacgagggtacgccagtttt

SEQ ID NO: 236 - DNA, 34 bp, Artificial Sequence
  synthesized seqence, 8s-2
acgtcaaagcgaacctcccgacttgcgggaggtt

SEQ ID NO: 237 - DNA, 40 bp, Artificial Sequence
  synthesized seqence, 65long
tcttaaacagcttgatgtgccgtcgagagggtgagccgcc

SEQ ID NO: 238 - DNA, 40 bp, Artificial Sequence
  synthesized seqence, 66shortTelo
gtatagccgctttcgaggtgaatttttttagggttagggt

SEQ ID NO: 239 - DNA, 40 bp, Artificial Sequence
  synthesized seqence, 69long
acaatgacaacaaccatagcggggttttgctccaggtcag

SEQ ID NO: 240 - DNA, 40 bp, Artificial Sequence
  synthesized seqence, 70shortTelo
gcggataaaccgatagttgcgccgtttttagggttagggt

SEQ ID NO: 241 - DNA, 40 bp, Artificial Sequence
  synthesized seqence, 73long
gatatattcggtcgctaggctgagactcctcacaaataaa

SEQ ID NO: 242 - DNA, 40 bp, Artificial Sequence
  synthesized seqence, 74shortTelo
attaggattcgcccacgcataacctttttagggttagggt

SEQ ID NO: 243 - DNA, 40 bp, Artificial Sequence
  synthesized seqence, 78s-telo
gtattaaggaggcttgcagggagttttttagggttagggt

SEQ ID NO: 244 - DNA, 40 bp, Artificial Sequence
  synthesized seqence, 166s-telo
gtcgaggtagagatagaacccttctttttagggttagggt

SEQ ID NO: 245 - DNA, 40 bp, Artificial Sequence
  synthesized seqence, 169long
ggacattctggccaacgccgtaaagcactaaaatcctgtt

SEQ ID NO: 246 - DNA, 40 bp, Artificial Sequence
  synthesized seqence, 170shortTelo
ctaaagggcgaccagtaataaaagtttttagggttagggt

SEQ ID NO: 247 - DNA, 40 bp, Artificial Sequence
  synthesized seqence, 173long
agattcaccagtcacaagcccccgatttagagcggtccac

SEQ ID NO: 248 - DNA, 40 bp, Artificial Sequence
  synthesized seqence, 174shortTelo
ggaaagccgattatttacattggctttttagggttagggt

SEQ ID NO: 249 - DNA, 40 bp, Artificial Sequence
  synthesized seqence, 177long
tcaatcgtctgaaatgggcgaacgtggcgagatcaccgcc

SEQ ID NO: 250 - DNA, 40 bp, Artificial Sequence
  synthesized seqence, 178shortTelo
gaagaaagacctacattttgacgctttttagggttagggt

SEQ ID NO: 251 - DNA, 40 bp, Artificial Sequence; identical to SEQ ID NO: 237
  synthesized seqence, 65long

SEQ ID NO: 252 - DNA, 40 bp, Artificial Sequence; identical to SEQ ID NO: 238
  synthesized seqence, 66shortTelo

SEQ ID NO: 253 - DNA, 40 bp, Artificial Sequence; identical to SEQ ID NO: 239
  synthesized seqence, 69long

SEQ ID NO: 254 - DNA, 40 bp, Artificial Sequence; identical to SEQ ID NO: 240
  synthesized seqence, 70shortTelo

SEQ ID NO: 255 - DNA, 54 bp, Artificial Sequence
  synthesized seqence, 73LongCompM20
gtgcaggtagttttgatatattcggtcgctaggctgagactcctcacaaataaa

SEQ ID NO: 256 - DNA, 40 bp, Artificial Sequence; identical to SEQ ID NO: 242
  synthesized seqence, 74shortTelo

SEQ ID NO: 257 - DNA, 40 bp, Artificial Sequence; identical to SEQ ID NO: 243
  synthesized seqence, 78s-telo

SEQ ID NO: 258 - DNA, 40 bp, Artificial Sequence; identical to SEQ ID NO: 244
  synthesized seqence, 166s-telo

SEQ ID NO: 259 - DNA, 56 bp, Artificial Sequence
  synthesized seqence, 169LongCompM20
gtgcaggtagttttggaacatttctggccaacgccgtaaagcactaaaatcctgtt

SEQ ID NO: 260 - DNA, 40 bp, Artificial Sequence; identical to SEQ ID NO: 246
  synthesized seqence, 170shortTelo

SEQ ID NO: 261 - DNA, 40 bp, Artificial Sequence; identical to SEQ ID NO: 247
  synthesized seqence, 173long

SEQ ID NO: 262 - DNA, 40 bp, Artificial Sequence; identical to SEQ ID NO: 248
  synthesized seqence, 174shortTelo

SEQ ID NO: 263 - DNA, 40 bp, Artificial Sequence; identical to SEQ ID NO: 249
  synthesized seqence, 177long

SEQ ID NO: 264 - DNA, 40 bp, Artificial Sequence; identical to SEQ ID NO: 250
  synthesized seqence, 178shortTelo

SEQ ID NO: 265 - DNA, 40 bp, Artificial Sequence
  synthesized seqence, 81cLong
caatatttttgaatggtataaacagttaatgcgtcataca

SEQ ID NO: 266 - DNA, 73 bp, Artificial Sequence
  synthesized seqence, 82sBothLongM20
gtgcaggtagtttttaaaggccgcttttgcgaatacgtggcacagattttctacctgcactataagcacttta

SEQ ID NO: 267 - DNA, 40 bp, Artificial Sequence
  synthesized seqence, 85cLong
cgcgaactgatagcccaacggggtcagtgcctactggtaa

SEQ ID NO: 268 - DNA, 51 bp, Artificial Sequence
  synthesized seqence, 86cShortMainM20
agtgcccgctattagtctttaatgttttctacctgcactataagcacttta

SEQ ID NO: 269 - DNA, 41 bp, Artificial Sequence
  synthesized seqence, 157cLong
aaacggctacagaggctacgtggactccaacgtagtccact

SEQ ID NO: 270 - DNA, 51 bp, Artificial Sequence
  synthesized seqence, 158cShortMainM20
gaaaaacctcggaacgagggtagcttttctacctgcactataagcacttta

SEQ ID NO: 271 - DNA, 40 bp, Artificial Sequence
  synthesized seqence, 161cLong
gcagcgaaagacagcagtctatcagggcgatgtagggttg

SEQ ID NO: 272 - DNA, 73 bp, Artificial Sequence
  synthesized seqence, 162sBothLongM20
gtgcaggtagtttttgacctgaaagcgtaagggatcgtcaccctcattttctacctgcactataagcacttta

SEQ ID NO: 273 - DNA, 7249 bp, Artificial Sequence
  synthesized seqence, M13mp18
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 274 - RNA, 23 nt, Homo sapiens
uaaagugcuuauagugcagguag

SEQ ID NO: 275 - DNA, 40 bp, Artificial Sequence; identical to SEQ ID NO: 237
  synthesized seqence, 65long

SEQ ID NO: 276 - DNA, 40 bp, Artificial Sequence; identical to SEQ ID NO: 238
  synthesized seqence, 66shortTelo

SEQ ID NO: 277 - DNA, 40 bp, Artificial Sequence; identical to SEQ ID NO: 239
  synthesized seqence, 69long

SEQ ID NO: 278 - DNA, 40 bp, Artificial Sequence; identical to SEQ ID NO: 240
  synthesized seqence, 70shortTelo

SEQ ID NO: 279 - DNA, 53 bp, Artificial Sequence
  synthesized seqence, 73LongCompCF509
cagttcgagttttgatatattcggtcgctaggctgagactcctcacaaataaa

SEQ ID NO: 280 - DNA, 40 bp, Artificial Sequence; identical to SEQ ID NO: 242
  synthesized seqence, 74shortTelo

SEQ ID NO: 281 - DNA, 40 bp, Artificial Sequence; identical to SEQ ID NO: 243
  synthesized seqence, 78s-telo

SEQ ID NO: 282 - DNA, 40 bp, Artificial Sequence; identical to SEQ ID NO: 244
  synthesized seqence, 166s-telo

SEQ ID NO: 283 - DNA, 55 bp, Artificial Sequence
  synthesized seqence, 169LongCompCF509
cagttcgagttttggaacatttctggccaacgccgtaaagcactaaaatcctgtt

SEQ ID NO: 284 - DNA, 40 bp, Artificial Sequence; identical to SEQ ID NO: 246
  synthesized seqence, 170shortTelo

SEQ ID NO: 285 - DNA, 40 bp, Artificial Sequence; identical to SEQ ID NO: 247
  synthesized seqence, 173long

SEQ ID NO: 286 - DNA, 40 bp, Artificial Sequence; identical to SEQ ID NO: 248
  synthesized seqence, 174shortTelo

SEQ ID NO: 287 - DNA, 40 bp, Artificial Sequence; identical to SEQ ID NO: 249
  synthesized seqence, 177long

SEQ ID NO: 288 - DNA, 40 bp, Artificial Sequence; identical to SEQ ID NO: 250
  synthesized seqence, 178shortTelo

SEQ ID NO: 289 - DNA, 40 bp, Artificial Sequence; identical to SEQ ID NO: 265
  synthesized seqence, 81cLong

SEQ ID NO: 290 - DNA, 68 bp, Artificial Sequence
  synthesized seqence, 82sBothLongCF509
cagttcgagtttttaaaggccgcttttgcgaatacgtggcacagattttctcgaactgcacctcatgg

SEQ ID NO: 291 - DNA, 40 bp, Artificial Sequence; identical to SEQ ID NO: 267
  synthesized seqence, 85cLong

SEQ ID NO: 292 - DNA, 47 bp, Artificial Sequence
  synthesized seqence, 86cShortCF509
agtgcccgctattagtctttaatgttttctcgaactgcacctcatgg

SEQ ID NO: 293 - DNA, 41 bp, Artificial Sequence; identical to SEQ ID NO: 269
  synthesized seqence, 157cLong

SEQ ID NO: 294 - DNA, 47 bp, Artificial Sequence
  synthesized seqence, 158cShortCF509
gaaaaacctcggaacgagggtagcttttctcgaactgcacctcatgg

SEQ ID NO: 295 - DNA, 40 bp, Artificial Sequence; identical to SEQ ID NO: 271
  synthesized seqence, 161cLong

SEQ ID NO: 296 - DNA, 68 bp, Artificial Sequence
  synthesized seqence, 162sBothLongCF509
cagttcgagtttttgacctgaaagcgtaagggatcgtcaccctcattttctcgaactgcacctcatgg

SEQ ID NO: 297 - DNA, 19 bp, Artificial Sequence
  synthesized seqence, R509
ccatgaggtgcagttcgag

SEQ ID NO: 298 - DNA, 40 bp, Artificial Sequence; identical to SEQ ID NO: 237
  synthesized seqence, 65long

SEQ ID NO: 299 - DNA, 40 bp, Artificial Sequence; identical to SEQ ID NO: 238
  synthesized seqence, 66shortTelo

SEQ ID NO: 300 - DNA, 40 bp, Artificial Sequence; identical to SEQ ID NO: 239
  synthesized seqence, 69long

SEQ ID NO: 301 - DNA, 40 bp, Artificial Sequence; identical to SEQ ID NO: 240
  synthesized seqence, 70shortTelo

SEQ ID NO: 302 - DNA, 54 bp, Artificial Sequence
  synthesized seqence, 73LongCompN1
aacattgccattttgatatattcggtcgctaggctgagactcctcacaaataaa

SEQ ID NO: 303 - DNA, 40 bp, Artificial Sequence; identical to SEQ ID NO: 242
  synthesized seqence, 74shortTelo

SEQ ID NO: 304 - DNA, 40 bp, Artificial Sequence; identical to SEQ ID NO: 243
  synthesized seqence, 78s-telo

SEQ ID NO: 305 - DNA, 40 bp, Artificial Sequence; identical to SEQ ID NO: 244
  synthesized seqence, 166s-telo

SEQ ID NO: 306 - DNA, 54 bp, Artificial Sequence
  synthesized seqence, 169LongCompN1
aacattgccattttggacattctggccaacgccgtaaagcactaaaatcctgtt

SEQ ID NO: 307 - DNA, 40 bp, Artificial Sequence; identical to SEQ ID NO: 246
  synthesized seqence, 170shortTelo

SEQ ID NO: 308 - DNA, 40 bp, Artificial Sequence; identical to SEQ ID NO: 247
  synthesized seqence, 173long

SEQ ID NO: 309 - DNA, 40 bp, Artificial Sequence; identical to SEQ ID NO: 248
  synthesized seqence, 174shortTelo

SEQ ID NO: 310 - DNA, 40 bp, Artificial Sequence; identical to SEQ ID NO: 249
  synthesized seqence, 177long

SEQ ID NO: 311 - DNA, 40 bp, Artificial Sequence; identical to SEQ ID NO: 250
  synthesized seqence, 178shortTelo

SEQ ID NO: 312 - DNA, 40 bp, Artificial Sequence; identical to SEQ ID NO: 265
  synthesized seqence, 81cLong

SEQ ID NO: 313 - DNA, 75 bp, Artificial Sequence
  synthesized seqence, 82sBothLongN1
aacattgccatttttaaaggccgcttttgcgaatacgtggcacagatttttggcaatgttgttccttgaggaagt

SEQ ID NO: 314 - DNA, 40 bp, Artificial Sequence; identical to SEQ ID NO: 267
  synthesized seqence, 85cLong

SEQ ID NO: 315 - DNA, 53 bp, Artificial Sequence
  synthesized seqence, 86cShortN1
agtgcccgctattagtctttaatgtttttggcaatgttgttccttgaggaagt

SEQ ID NO: 316 - DNA, 41 bp, Artificial Sequence; identical to SEQ ID NO: 269
  synthesized seqence, 157cLong

SEQ ID NO: 317 - DNA, 53 bp, Artificial Sequence
  synthesized seqence, 158cShortN1
gaaaaacctcggaacgagggtagctttttggcaatgttgttccttgaggaagt

SEQ ID NO: 318 - DNA, 40 bp, Artificial Sequence; identical to SEQ ID NO: 271
  synthesized seqence, 161cLong

SEQ ID NO: 319 - DNA, 75 bp, Artificial Sequence
  synthesized seqence, 162sBothLongN1
aacattgccatttttgacctgaaagcgtaagggatcgtcaccctcatttttggcaatgttgttccttgaggaagt

SEQ ID NO: 320 - DNA, 25 bp, Artificial Sequence
  synthesized seqence, N1
acttcctcaaggaacaacattgcca

SEQ ID NO: 321 - DNA, 40 bp, Artificial Sequence; identical to SEQ ID NO: 237
  synthesized seqence, 65long

SEQ ID NO: 322 - DNA, 40 bp, Artificial Sequence; identical to SEQ ID NO: 238
  synthesized seqence, 66shortTelo

SEQ ID NO: 323 - DNA, 40 bp, Artificial Sequence; identical to SEQ ID NO: 239
  synthesized seqence, 69long

SEQ ID NO: 324 - DNA, 40 bp, Artificial Sequence; identical to SEQ ID NO: 240
  synthesized seqence, 70shortTelo

SEQ ID NO: 325 - DNA, 54 bp, Artificial Sequence
  synthesized seqence, 73LongCompHD1T1
acaccaacctttttgatatattcggtcgctaggctgagactcctcacaaataaa

SEQ ID NO: 326 - DNA, 40 bp, Artificial Sequence; identical to SEQ ID NO: 242
  synthesized seqence, 74shortTelo

SEQ ID NO: 327 - DNA, 40 bp, Artificial Sequence; identical to SEQ ID NO: 243
  synthesized seqence, 78s-telo

SEQ ID NO: 328 - DNA, 40 bp, Artificial Sequence; identical to SEQ ID NO: 244
  synthesized seqence, 166s-telo

SEQ ID NO: 329 - DNA, 54 bp, Artificial Sequence
  synthesized seqence, 169LongCompHD1T1
acaccaacctttttggacattctggccaacgccgtaaagcactaaaatcctgtt

SEQ ID NO: 330 - DNA, 40 bp, Artificial Sequence; identical to SEQ ID NO: 246
  synthesized seqence, 170shortTelo

SEQ ID NO: 331 - DNA, 40 bp, Artificial Sequence; identical to SEQ ID NO: 247
  synthesized seqence, 173long

SEQ ID NO: 332 - DNA, 40 bp, Artificial Sequence; identical to SEQ ID NO: 248
  synthesized seqence, 174shortTelo

SEQ ID NO: 333 - DNA, 40 bp, Artificial Sequence; identical to SEQ ID NO: 249
  synthesized seqence, 177long

SEQ ID NO: 334 - DNA, 40 bp, Artificial Sequence; identical to SEQ ID NO: 250
  synthesized seqence, 178shortTelo

SEQ ID NO: 335 - DNA, 40 bp, Artificial Sequence; identical to SEQ ID NO: 265
  synthesized seqence, 81cLong

SEQ ID NO: 336 - DNA, 71 bp, Artificial Sequence
  synthesized seqence, 82sBothLongHD1T1
acaccaaccttttttaaaggccgcttttgcgaatacgtggcacagatttgtggttggtgtggttggtcata

SEQ ID NO: 337 - DNA, 40 bp, Artificial Sequence; identical to SEQ ID NO: 267
  synthesized seqence, 85cLong

SEQ ID NO: 338 - DNA, 57 bp, Artificial Sequence
  synthesized seqence, 86cShortHD1T1
agtgcccgctattagtctttaatgttttagtccgtggtagggcaggttggggtgact

SEQ ID NO: 339 - DNA, 41 bp, Artificial Sequence; identical to SEQ ID NO: 269
  synthesized seqence, 157cLong

SEQ ID NO: 340 - DNA, 57 bp, Artificial Sequence
  synthesized seqence, 158cShortHD1T1
gaaaaacctcggaacgagggtagcttttagtccgtggtagggcaggttggggtgact

SEQ ID NO: 341 - DNA, 40 bp, Artificial Sequence; identical to SEQ ID NO: 271
  synthesized seqence, 161cLong

SEQ ID NO: 342 - DNA, 71 bp, Artificial Sequence
  synthesized seqence, 162sBothLongHD1T1
acaccaaccttttttgacctgaaagcgtaagggatcgtcaccctcatttgtggttggtgtggttggtcata

SEQ ID NO: 343 - DNA, 25 bp, Artificial Sequence
  synthesized seqence, HD1
tttgtggttggtgtggttggtcata

SEQ ID NO: 344 - DNA, 20 bp, Artificial Sequence
  synthesized seqence, R29142
gttcctggtccccaaaattt

SEQ ID NO: 345 - DNA, 40 bp, Artificial Sequence; identical to SEQ ID NO: 237
  synthesized seqence, 65long

SEQ ID NO: 346 - DNA, 40 bp, Artificial Sequence; identical to SEQ ID NO: 238
  synthesized seqence, 66shortTelo

SEQ ID NO: 347 - DNA, 40 bp, Artificial Sequence; identical to SEQ ID NO: 239
  synthesized seqence, 69long

SEQ ID NO: 348 - DNA, 40 bp, Artificial Sequence; identical to SEQ ID NO: 240
  synthesized seqence, 70shortTelo

SEQ ID NO: 349 - DNA, 54 bp, Artificial Sequence; identical to SEQ ID NO: 255
  synthesized seqence, 73LongCompM20

SEQ ID NO: 350 - DNA, 40 bp, Artificial Sequence; identical to SEQ ID NO: 242
  synthesized seqence, 74shortTelo

SEQ ID NO: 351 - DNA, 40 bp, Artificial Sequence; identical to SEQ ID NO: 243
  synthesized seqence, 78s-telo

SEQ ID NO: 352 - DNA, 40 bp, Artificial Sequence; identical to SEQ ID NO: 244
  synthesized seqence, 166s-telo

SEQ ID NO: 353 - DNA, 56 bp, Artificial Sequence; identical to SEQ ID NO: 259
  synthesized seqence, 169LongCompM20

SEQ ID NO: 354 - DNA, 40 bp, Artificial Sequence; identical to SEQ ID NO: 246
  synthesized seqence, 170shortTelo

SEQ ID NO: 355 - DNA, 40 bp, Artificial Sequence; identical to SEQ ID NO: 247
  synthesized seqence, 173long

SEQ ID NO: 356 - DNA, 40 bp, Artificial Sequence; identical to SEQ ID NO: 248
  synthesized seqence, 174shortTelo

SEQ ID NO: 357 - DNA, 40 bp, Artificial Sequence; identical to SEQ ID NO: 249
  synthesized seqence, 177long

SEQ ID NO: 358 - DNA, 40 bp, Artificial Sequence; identical to SEQ ID NO: 250
  synthesized seqence, 178shortTelo

SEQ ID NO: 359 - DNA, 40 bp, Artificial Sequence; identical to SEQ ID NO: 265
  synthesized seqence, 81cLong

SEQ ID NO: 360 - DNA, 73 bp, Artificial Sequence; identical to SEQ ID NO: 266
  synthesized seqence, 82sBothLongM20

SEQ ID NO: 361 - DNA, 40 bp, Artificial Sequence; identical to SEQ ID NO: 267
  synthesized seqence, 85cLong

SEQ ID NO: 362 - DNA, 51 bp, Artificial Sequence; identical to SEQ ID NO: 268
  synthesized seqence, 86cShortMainM20

SEQ ID NO: 363 - DNA, 41 bp, Artificial Sequence; identical to SEQ ID NO: 269
  synthesized seqence, 157cLong

SEQ ID NO: 364 - DNA, 51 bp, Artificial Sequence; identical to SEQ ID NO: 270
  synthesized seqence, 158cShortMainM20

SEQ ID NO: 365 - DNA, 40 bp, Artificial Sequence; identical to SEQ ID NO: 271
  synthesized seqence, 161cLong

SEQ ID NO: 366 - DNA, 73 bp, Artificial Sequence; identical to SEQ ID NO: 272
  synthesized seqence, 162sBothLongM20

SEQ ID NO: 367 - DNA, 16 bp, Artificial Sequence
  synthesized seqence, SmBiT-95w
aaggaattgaggaagg

SEQ ID NO: 368 - DNA, 16 bp, Artificial Sequence
  synthesized seqence, LgBiT-138w
atccgcgacctgctcc